SEQUENCE LISTING (6 sequences)

SEQ ID NO: 1 - RNA, 21 nt, Artificial Sequence
  Description of Artificial Sequence Synthetic oligonucleotide
gaucaagagacuccucaguuu

SEQ ID NO: 2 - RNA, 21 nt, Artificial Sequence
  Description of Artificial Sequence Synthetic oligonucleotide
acugaggagucucuugaucuu

SEQ ID NO: 3 - DNA, 21 bp, Artificial Sequence
  Description of Combined DNA/RNA Molecule Synthetic oligonucleotide
cguacgcggaauacuucgatt

SEQ ID NO: 4 - DNA, 21 bp, Artificial Sequence
  Description of Combined DNA/RNA Molecule Synthetic oligonucleotide
ucgaaguauuccgcguacgtt

SEQ ID NO: 5 - DNA, 21 bp, Artificial Sequence
  Description of Combined DNA/RNA Molecule Synthetic oligonucleotide
cguacgcggaauacuucgatt

SEQ ID NO: 6 - DNA, 21 bp, Artificial Sequence
  Description of Combined DNA/RNA Molecule Synthetic oligonucleotide
ucgaaguauuccgcguacgtt

(19) United States
(12) Patent Application Publication
(10) Pub. No.: US 2009/0232730 A1
(43) Pub. Date: Sep. 17, 2009

(54) METHOD OF PRODUCING IMMUNOLIPOSOMES AND COMPOSITIONS THEREOF

(75) Inventors: Dan Peer, Brookline, MA (US); Motomu Shimaoka, Brookline, MA (US)

Correspondence Address: DAVID S. RESNICK, NIXON PEABODY LLP, 100 SUMMER STREET, BOSTON, MA, 02110-2131, US

(73) Assignee: IMMUNE DISEASE INSTITUTE, INC., Boston, MA (US)

(21) Appl. No.: 12/298,181

(22) PCT Filed: Apr. 24, 2007
(86) PCT No.: PCT/US07/10075

§ 371 Date: Oct. 23, 2008

(30) Foreign Application Priority Data
Apr. 24, 2006 (US) 60794361

(51) Int. Cl. A61K 51/00; A61K 9/127; A61K 31/7088; A61K 39/395; A61B 5/055

(52) U.S. Cl. 424/1.21; 424/450; 514/44 R; 424/130.1; 424/9.3

(57) ABSTRACT

The invention provides a method for a multi-layered lipid particles in the form of liposomes that are coated first with a cryoprotectant followed by a targeting moiety over the coat of cryoprotectant, and a method for encapsulating drugs and agents in the multi-layered coated liposomes. In addition, ready-to-use liposome kits for coating with targeting agent of choice and for drug and/or agent encapsulation.

### CROSS REFERENCE TO RELATED APPLICATIONS

This application claims priority to U.S. Provisional Application No. 60/794,361 filed Apr. 24, 2006, the contents of which are incorporated entirely herein by reference.

### GOVERNMENT SUPPORT

This invention was supported by R01 AI063421 awarded by the National Institutes of Health (NIH). The government has certain rights in the invention.

### BACKGROUND OF THE INVENTION

Liposomes, spherical, self-enclosed vesicles composed of amphipathic lipids, have been widely studied and are employed as vehicles for in vivo administration of therapeutic agents. In particular, the so-called long circulating liposomes formulations which avoid uptake by the organs of the mononuclear phagocyte system, primarily the liver and spleen, have found commercial applicability. Such long-circulating liposomes include a surface coat of flexible water soluble polymer chains, which act to prevent interaction between the liposome and the plasma components which play a role in liposome uptake. Alternatively, hyaluronan has been used as a surface coating to maintain long circulation.

More recently, efforts have focused on ways to achieve site specific delivery of long-circulating liposomes. In one approach, targeting ligands, such as an antibody, are attached to the liposomes' surfaces. This approach, where the targeting ligand is bound to the polar head group residues of liposomal lipid components, results in interference by the surface-grafted polymer chains, inhibiting the interaction between the bound ligand and its intended target (Klibanov, A. L., et al., Biochim. Biophys. Acta., 1062: 142-148 (1991); Hansen, C. B., et al., Biochim. Biophys. Acta, 1239: 133-144 (1995)).

In another approach, the targeting ligand is attached to the free ends of the polymer chains forming the surface coat on the liposomes (Allen. T. M., et al., Biochim. Biophys. Acta, 1237: 99-108 (1995); Blume, G., et al., Biochim. Biophys. Acta, 1149: 180-184 (1993)). Two approaches have been described for preparing a liposome having a targeting ligand attached to the distal end of the surface polymer chains. One approach involves preparation of lipid vesicles which include an end-functionalized lipid-polymer derivative; that is, a lipid-polymer conjugate where the free polymer end is reactive or “activated”. Such an activated conjugate is included in the liposome composition and the activated polymer ends are reacted with a targeting ligand after liposome formation. The disadvantage to this approach is the difficulty in reacting all of the activated ends with a ligand. The approach also requires a subsequent step for separation of the unreacted ligand from the liposome composition.

In another approach, the lipid-polymer-ligand conjugate is included in the lipid composition at the time of liposome formation. This approach has the disadvantage that some of the valuable ligand faces the inner aqueous compartment of the liposome and is unavailable for interaction with the intended target.

In another approach, cryoprotectants have been included in the liposome composition. Smaller liposomes tend to relapse into larger liposomes or leak the contents of the vesicles, particularly during lyophiliation and rehydration. Most frequently, sugars, such as trehalose, sucrose, mannose or glucose have been used. However, high concentrations of the sugars are necessary and administration of the sugars may be detrimental to the subject. More recently, hyaluronan has been shown to be useful as a cryoprotectant, as well as a targeting agent.

These approaches suffer from a lack of flexibility in designing a therapeutic composition that is specific for a target cell for a specific patient. There is then a need for a liposome composition which provides flexibility in choice of the entrapped agent and the targeting ligand, while also providing for long circulation.

### SUMMARY OF THE INVENTION

The present invention provides a method for layer by layer coating of lipid particles with a cryoprotectant and a targeting moiety. In the embodiment, the method comprises the steps of (1) providing a lipid particle having phospholipids conjugated to a cryoprotectant, wherein the cryoprotectant has a functional group; (2) crosslinking the targeting moiety to the cryoprotectant by way of the functional group on the cryoprotectant; (3) lyophilizing the lipid particle having the cryoprotectant and the targeting moiety crosslinked to it; (4) providing an aqueous solution of an agent of interest; and (5) rehydrating the lyophilized lipid particle having the cryoprotectant and the targeting moiety crosslinked to it with the aqueous solution of the agent.

In one embodiment, the present invention provides a lipid particle coated with a cryoprotectant. In another embodiment, a targeting moiety is linked to the cryoprotectant. In one embodiment, the lipid particle comprises a phospholipid with a functional group, wherein the functional group may be crosslinked to the cryoprotectant, wherein the cryoprotectant is covalently bound to the functional group of the phospholipid. In one embodiment, the targeting moiety is covalently bound to the cryoprotectant.

The lipid particle may be a liposome. The lipid particle may be a micelle.

The cryoprotectant may be a glycosaminoglycan, e.g., hyaluronan. Other cryoprotectants include disaccharide and monosaccharide sugars such as trehalose, maltose, sucrose, maltose, fructose, glucose, lactose, saccharose, galactose, mannose, xylit and sorbit, mannitol, dextran; polyols such as glycerol, glycerin, polyglycerin, ethylene glycol, prolylene glycol, polyethyleneglycol; aminoglycosides; and dimethylsulfoxide.

The lipid particle may comprise a hydrophobic agent in the lipid layer, e.g., associated with the lipids of the lipid particle.

The lipid particle may comprise a hydrophilic agent encapsulated within.

The agent may be a nucleic acid, such as plasmid DNA, short interfering RNA (siRNA), short-hairpin RNA, small temporal RNA (stRNA), microRNA (miRNA), RNA mimetics, or heterochromatic siRNA condensed with a cationic peptide, such as a protamine sulfate and polylysine or a cationic polymer, such as polyethyleneimine (PEI), polyamine spermidine, and spermine.

The functional group on the phospholipids wherein the cryoprotectant is crosslinked may be an amine group or a carboxyl group.

The present invention provides a method for encapsulating agents in a lipid particle. The method comprises the steps of: (1) providing a lyophilized lipid particle having phospholipids conjugated with a cryoprotectant, wherein the cryoprotectant is covalently linked to the phospholipid, and a targeting moiety is covalently linked to the cryoprotectant; (2) providing a hydrophilic agent in aqueous solution; and (3) rehydrating the lyophilized lipid particle with the aqueous solution comprising the hydrophilic agent. The lipid particle may be lyophilized from an aqueous solution, such as a buffer solution, similar to that used for the hydrophilic agent. The aqueous solution may be water.

The present invention also provides ready-to-use liposome kits for coating with targeting agent of choice and for drug and/or agent encapsulation.

### BRIEF DESCRIPTION OF THE DRAWINGS

FIGS. 1A-1C show hyaluronan as a cryoprotectant. FIG. 1A shows a dashed curve which represents binding of the nanoliposomes coated with CBRM1/29 (ULV-CBRM1/29-cy3) to K562 cells expressing Mac-1 integrin before lyophilization. The solid curve represents the binding of the carrier after lyophilization. As shown in this panel, mean fluorescence intensity (MFI) before lyophilization was 175, and after lyophilization and reconstitution, 5.6. FIG. 1B shows an almost complete overlay of the dashed (MFI=161, before lyophilization) and solid (MFI=172, after lyophilization and reconstitute) curves of tHA-CBRM1/29-cy3 carriers. These carriers have HA as a cryoprotectant on their first layer, preserving their structure during lyophilization and reconstitution. FIG. 1C shows a complete overlay between the curves on the antibody itself, CBRM1/29-cy3 (before (MFI=82) and after (MFI=73) lyophilization and reconstitute), showing that the changes in particles size are the major factor for changes in binding capacity to these cells.

FIG. 2 shows the encapsulation and sustained release properties of HA-nanoliposomes having on their surface either CBRM1/29 antibody against integrin Mac-1 (tHA-CBRM1/29) or Her2 scFv against ErbB2 receptor (tHA-Her2).

FIGS. 3A-3B show the results of an in vitro cytotoxicity assay (4/48 h) in Mac-1 cells. FIG. 3A shows all three formulations (free MMC, MMC entrapped in nano-scale HA-coated liposomes, named ULV-HA, and in antibody-coated HA-coated nanoliposomes (tHA-CBRM1/29)), in K562 mock cells. FIG. 3B shows that where cells expressed integrin Mac-1, only the cells that got the liposomal formulation with the antibody had dramatic decrease in cell survival

FIGS. 4A-4B show binding of the nanoliposomes with immobilized CBRM1/5-cy3 on the surface to an active isolated primary human neutrophils (activation by TNF-α), or without activation. tHA-CBRM1/5-cy3 is bound to primary isolated human neutrophils only upon activation of the cells with TNF-α (FIG. 4A), whereas tHA-IgG1 mouse control isotype did not give any binding to these cells. Similar trends have been observed with other cells such as K562 cells stable transfected with Mac-1 integrin and activated by PMA (FIG. 4B).

FIGS. 5A-5B show tHA-IgG57 and tHA-TS1/22 targeting active integrin LFA-1 in human primary CD4+ cells. FIG. 5A shows binding of IgG57 (tHA-IgG57) in an active/non active form of another integrin, LFA-1, in primary human lymphocytes compare to non binding curve by tHA-IgG1 (human isotype control). FIG. 5B shows binding with another antibody (TS1/22) on the surface of nanoliposomes. This antibody recognizes both active and non active conformation of integrin LFA-1 that are overlay one on the other. As a negative control, a tHA-IgG1 mouse isotype control has been used.

FIG. 6 shows a flow cytometry analysis of Mac-1 WT cells with siRNA-cy3 alone, siRNA-cy3 transfected using linear PEI and siRNA-cy3 condensed by linear PEI and entrapped inside tHA-CBRM1/5 that target active integrin Mac-1.

FIG. 7 shows flow cytometry analysis of siRNA-cy3 condensed by protamine and entrapped in tHA-CBRM1/5 in primary human neutrophils with or without activation of the cells.

FIG. 8 shows the release profile of siRNA-cy3 from the various carriers incubated for 20 hours in 50% human serum.

FIG. 9 shows selective silencing of CD4+ cells using tHA-TS1/22 that target integrin LFA-1 on these cells.

FIGS. 10A-10B show silencing of CD4 with the specific conformation-specific carrier tHA-IgG57. FIG. 10A shows effective silencing in the same cells with a different delivery system targeting only active integrin (tHA-IgG57). A dose response of siRNA-CD4 is shown in FIG. 10B.

FIG. 11 shows CD4 silencing in physiological conditions, in an in vitro inflammation model.

FIG. 12 shows CD4 silencing in human primary CD4+ cells via immunomicelles.

### DETAILED DESCRIPTION OF THE INVENTION

The term “lipid particle” refers to lipid vesicles such as liposomes or micelles.

Many efforts have been made over the years to coat particles with long circulating agents (Cattel et al. J Chemother. 2004; Suppl 4:94-7; Laverman et al. Crit Rev Ther Drug Carrier Syst. 2001; 18:551-66; Gref et al. Science. 1994; 263:1600-3). The first generation comprised the particles themselves. The second generation included targeting agents that were directly immobilized on the surface of the particles (Park et al. Semin Oncol. 2004; 31 (6 Suppl 13):196-205; Olivier et al. NeuroRx. 2005; 2:108-19), and the third generation included both long-circulating agents that were first conjugated to a targeter and then directly immobilized on the particle's surface (Sapra et al. Clin Cancer Res. 2004; 10:1100-11; Allen et al. J Liposome Res. 2002; 12:5-12; Maruyama et al. FEBS Lett. 1997 11; 413:177-80). Examples of long-circulating agents include mostly PEG and hyaluronan. Other cryoprotectants sugars include: sucrose, trihalose, mannose, and recently also hyaluronan (Peer et al. Biochim. Biophys. Acta. 2003; 1612: 76-82).

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

Here we describe our invention of a simple, straightforward method for coating small lipid particles, e.g., liposomes or micelles, layer-by-layer with a first layer of a cryoprotectant and a second layer of a targeting agent, e.g., antibody, scFv, or a receptor ligand. We further describe our invention of a method for encapsulating agents in a particle having both a cryoprotectant and a targeter.

We have invented a method in which micro/nano-lipid particles, e.g, liposomes, spheres, micelles, are coated with a first layer containing agents that facilitate cryoprotection, long half-life in circulation, or both (PEG, hyaluronan, others), and, after purification, if necessary, are coated with a second layer containing targeting agents, e.g., specific monoclonal antibodies, scFvs, Fab fragments, or receptor ligands. Then, virtually any drug can be encapsulated in the carriers via lyophilization and reconstitution with an agent suspended in aqueous solution.

In one embodiment, the invention provides a method of coating a lipid particle that is pre-conjugated with a cryoprotectant, wherein the cryoprotectant has a functional group attached. The attached functional group may be activated and a targeting agent is crosslinked to the activated functional group to form a two-layer coated lipid particle which can then be lyophilized for storage purposes prior to use for drug or agent encapsulation.

In one embodiment, the invention is directed to a method to generate immunoliposomes wherein the composition includes both a cryoprotectant and a targeting agent. The targeting agents include, for example, an antibody or the antigen binding fragments thereof. Targeting moieties can selectively target leukocyte cells by specifically binding integrins that are exclusively or preferentially expressed on leukocytes. They can target activated leukocytes by targeting the leukocyte specific integrins in their active conformation. In one embodiment, the invention provides liposomes that may be stored in a lyophilized condition prior to encapsulation of drug or prior to addition of the targeting agent.

Encompassed in the invention is a liposome kit comprising ready-to-used lyophilized cryoprotectant-conjugated lipid particles with activated functional groups on the cryoprotectant for crosslinking to user selected targeting agent. The targeting agent coated lipid particle will then be ready for drug or agent encapsulation.

Encompassed in the invention is a liposome kit comprising ready-to-used lyophilized cryoprotectant-cum-targeting agent-conjugated lipid particles. For example, the lipid particle of the kit may be conjugated with antibodies against MUC1, MUC2, or MUC3 for targeting to tumors of breast, lung, and prostate cancers. Alternately, the lipid particle of the kit may be conjugated with antibodies against ganglioside GM3 for targeting to melanoma. The lyophilized lipid particle of the kit can be rehydrated directly in the drug or agent solution for drug or agent encapsulation respectively. The targeting agent may be functional fragments of an antibody.

In one embodiment, the invention is directed to immunoliposomes, and the method to generate such immunoliposomes, wherein the immunoliposomes are loaded with two agents. In one embodiment, one of the two agents is hydrophilic, and is encapsulated by the liposome. In another embodiment, the other agent is hydrophobic and is associated with the lipid layer of the liposome.

In one embodiment, the invention is directed to a method to encapsulate nucleic acids, e.g., plasmid DNA, DNA fragments, short interfering RNA (siRNA), short-hairpin RNA, small temporal RNA (stRNA), microRNA (miRNA), RNA mimetics, or heterochromatic siRNA. In one embodiment, The nucleic acids are condensed with a cationic polymer, e.g., PEI, polyamine spermidine, and spermine, or a cationic peptide, e.g., protamine and polylysine, and encapsulated in the lipid particle.

The present invention is directed to liposomes comprising multiple layers assembled in a step-wise fashion. In one embodiment, the first step is the preparation of empty nano-scale liposomes. Liposomes may be prepared by any method known to the skilled artisan. The second step is the addition of a first layer of surface modification. The first layer is added to the liposome by covalent modification. The first layer comprises hyaluronic acid, or other cryoprotectant glucosaminoglycan. The liposome composition may also be lyophilized and reconstituted at any time after the addition of the first layer. The third step is to add a second surface modification. The second layer is added by covalent attachment to the first layer. The second layer comprises a targeting agent, e.g., an antibody or functional fragment thereof. Further layers may add to the liposome and these layers may include additional targeting agents. Alternatively, the second layer may include a heterogeneous mix of targeting agents. The liposome composition is lyophilized after addition of the final targeting layer. The agent of interest is encapsulated by the liposome by rehydration of the liposome with an aqueous solution containing the agent. In one embodiment, agents that are poorly soluble in aqueous solutions or agents that are hydrophobic may be added to the composition during preparation of the liposomes in step one.

In another embodiment, the multi-layered liposomes of the invention is made with cryoprotectant conjugated lipid particles. The cryoprotectant is covalently linked to the lipid polar groups of the phospholipids and it forms the first layer of surface modification on the liposome discussed supra. The targeting agent forms the second layer of coat and it is added on to the first layer of cryoprotectant. The multi-layered liposome may be lyophilized for storage. The agent of interest is encapsulated by the liposome by rehydration of the liposome with an aqueous solution containing the agent.

### Liposomes

Liposomes are completely closed lipid bilayer membranes containing an entrapped aqueous volume. Liposomes may be unilamellar vesicles possessing a single membrane bilayer or multilameller vesicles, onion-like structures characterized by multiple membrane bilayers, each separated from the next by an aqueous layer. In one preferred embodiment, the liposomes of the present invention are unilamellar vesicles. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes according to the invention may be produced from combinations of lipid materials well known and routinely utilized in the art to produce liposomes. Lipids may include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

Liposomes of the present invention may be obtained by any method known to the skilled artisan. For example, the liposome preparation of the present invention can be produced by reverse phase evaporation (REV) method (see U.S. Pat. No. 4,235,871), infusion procedures, or detergent dilution. A review of these and other methods for producing liposomes may be found in the text Liposomes, Marc Ostro, ed., Marcel Dekker, Inc., New York, 1983, Chapter 1. See also Szoka Jr. et al., (1980, Ann. Rev. Biophys. Bioeng., 9:467). A method for forming ULVs is described in Cullis et al., PCT Publication No. 87/00238, Jan. 16, 1986, entitled “Extrusion Technique for Producing Unilamellar Vesicles”. Multilamellar liposomes (MLV) may be prepared by the lipid-film method, wherein the lipids are dissolved in a chloroform-methanol solution (3:1, vol/vol), evaporated to dryness under reduced pressure and hydrated by a swelling solution. Then, the solution is subjected to extensive agitation and incubation, e.g., 2 hour, e.g., at 37° C. After incubation, unilamellar liposomes (ULV) are obtained by extrusion. The extrusion step modifies liposomes by reducing the size of the liposomes to a preferred average diameter. Alternatively, liposomes of the desired size may be selected using techniques such as filtration or other size selection techniques. While the size-selected liposomes of the invention should have an average diameter of less than about 300 nm, it is preferred that they are selected to have an average diameter of less than about 200 nm with an average diameter of less than about 100 nm being particularly preferred. When the liposome of the present invention is a unilamellar liposome, it preferably is selected to have an average diameter of less than about 200 nm. The most preferred unilamellar liposomes of the invention have an average diameter of less than about 100 nm. It is understood, however, that multivesicular liposomes of the invention derived from smaller unilamellar liposomes will generally be larger and may have an average diameter of about less than 1000 nm. Preferred multivesicular liposomes of the invention have an average diameter of less than about 800 nm, and less than about 500 nm while most preferred multivesicular liposomes of the invention have an average diameter of less than about 300 nm.

In addition, in order to prevent the uptake of the liposomes into the cellular endothelial systems and enhance the uptake of the liposomes into the tissue of interest, the outer surface of the liposomes may be modified with a long-circulating agent. The modification of the liposomes with a hydrophilic polymer as the long-circulating agent is known to enable to prolong the half-life of the liposomes in the blood. Examples of the hydrophilic polymer include polyethylene glycol, polymethylethylene glycol, polyhydroxypropylene glycol, polypropylene glycol, polymethylpropylene glycol and polyhydroxypropylene oxide. One preferred hydrophilic polymer is polyethylene glycol (PEG). Glycosaminoglycans, e.g., hyaluronic acid, may also be used as long-circulating agents.

### Layer 1: Cryoprotectant

The term “cryoprotectant” refers to an agent that protects a lipid particle subjected to dehydration-rehydration, freeze-thawing, or lyophilization-rehydration from vesicle fusion and/or leakage of vesicle contents. Useful cryoprotectants in the methods of the present invention include hyaluronan/hyaluronic acid (HA) or other glycosaminoglycans for use with liposomes or micelles or PEG for use with micelles. Other cryoprotectants, but are not limited to, include disaccharide and monosaccharide sugars such as trehalose, maltose, sucrose, maltose, fructose, glucose, lactose, saccharose, galactose, mannose, xylit and sorbit, mannitol, dextran; polyols such as glycerol, glycerin, polyglycerin, ethylene glycol, prolylene glycol, polyethyleneglycol and branched polymers thereof; aminoglycosides; and dimethylsulfoxide.

The liposome preparation of the present invention is characterized in that it is further derivatized with a cryoprotectant. One preferred cryoprotectant of the present invention is hyaluronic acid or hyaluran (HA). Hyaluronic acid, a type of glycosaminoglycan, is a natural polymer with alternating units of N-acetyl glucosamine and glucoronic acid. Using a crosslinking reagent, hyaluronic acid offers carboxylic acid residues as functional groups for covalent binding. The N-acetyl-glucosamine contains hydroxyl units of the type —CH₂—OH which can be oxidized to aldehydes, thereby offering an additional method of crosslinking hyaluronic acid to the liposomal surface in the absence of a crosslinking reagent. Alternatively, other glycosaminoglycans, e.g., chondroitin sulfate, dermatan sulfate, keratin sulfate, or heparin, may be utilized in the methods of the present invention. Cryoprotectants are bound covalently to discrete sites on the liposome surfaces. The number and surface density of these sites will be dictated by the liposome formulation and the liposome type.

In one embodiment, the final ratio of cryoprotectant (μg) to lipid (μmole) is about 50 μg/μmole, about 55 μg/μmole, about 60 μg/μmole, about 65 μg/μmole, about 70 μg/μmole, about 75 μg/μmole, about 80 μg/μmole, about 85 μg/μmole, about 90 μg/μmole, about 95 μg/μmole, about 100 μg/μmole, about 105 μg/μmole, about 120 μg/mole, about 150 μg/mole, or about 200 μg/mole. In one embodiment, the ratio of cryoprotectant (μg) to lipid (μmole) is a range from 3-200 μg per mole lipid.

To form covalent conjugates of cryoprotectants and liposomes, crosslinking reagents have been studied for effectiveness and biocompatibility. Crosslinking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the chemistry of crosslinking, linkage of the amine residues of the recognizing substance and liposomes is established. Covalent attachment of the cryoprotectant HA is described in U.S. Pat. No. 5,846,561.

Subsequent to the covalent addition of the cryoprotectant, the lipid particles may be lyophilized. The lyophilized lipid particles may be rehydrated and the targeting agent (layer 2) covalently attached to the lipid particle. Alternatively, the targeting agent may be covalently attached to the lipid particle without prior lyophilization and rehydration.

### Layer 2: Targeting Agent

The term “targeting agent” or “targeting moiety” refers to an agent that homes in on or preferentially associates or binds to a particular tissue, cell type, receptor, infecting agent or other area of interest. Examples of a targeting agent include, but are not limited to, an oligonucleotide, an antigen, an antibody or functional fragment thereof, a ligand, a receptor, one member of a specific binding pair, a polyamide including a peptide having affinity for a biological receptor, an oligosaccharide, a polysaccharide, a steroid or steroid derivative, a hormone, e.g., estradiol or histamine, a hormone-mimic, e.g., morphine, or other compound having binding specificity for a target. In the methods of the present invention, the targeting agent promotes transport or preferential localization of the lipid particle of the present invention to the target of interest.

As used herein, an “antibody” or “functional fragment” of an antibody encompasses polyclonal and monoclonal antibody preparations, as well as preparations including hybrid or chimeric antibodies, such as humanized antibodies, altered antibodies, F(ab′)₂ fragments, F(ab) fragments, Fv fragments, single domain antibodies, dimeric and trimeric antibody fragment constructs, minibodies, and functional fragments thereof which exhibit immunological binding properties of the parent antibody molecule and/or which bind a cell surface antigen.

The targeting agent can be any ligand the receptor for which is differentially expressed on the target cell. Non-limiting examples include transferrin, folate, other vitamins, EGF, insulin, Heregulin, RGD peptides or other polypeptides reactive to integrin receptors, antibodies or their fragments. Sugar molecules or glycoproteins, lipid molecules or lipoproteins may be targeting agents.

In one embodiment, antibodies against cell surface markers that are specifically expressed in disease states can be used as targeting agent. Examples of antigens that specifically appear in tumors cells include ganglioside GM3 on melanoma, MUC1, MUC2, and MUC3 on the surface of breast cancer, lung cancer and prostate cancer, and Lewis X on the surface of gastro-intestinal digestive cancer. In one embodiment the antibody is a functional fragment containing the antigen binding region of the antibody. A preferred antibody fragment is a single chain Fv fragment of an antibody. The antibody or antibody fragment is one which will bind to a receptor on the surface of the target cell, and preferably to a receptor that is differentially expressed on the target cell. In one embodiment, multiple types of targeting agents may be covalently attached to the lipid particle.

The targeting agent is covalently conjugated to the cryoprotectant, e.g., HA. Crosslinking reagents include glutaraldehyde (GAD), bifunctional oxirane (OXR), ethylene glycol diglycidyl ether (EGDE), N-hydroxysuccinimide (NHS), and a water soluble carbodiimide, preferably 1-ethyl-3-(3-dimethylaminopropyl) carbodiimide (EDC). As is known to the skilled artisan, any crosslinking chemistry can be used, including, but not limited to, thioether, thioester, malimide and thiol, amine-carboxyl, amine-amine, and others listed in organic chemistry manuals, such as, Elements of Organic Chemistry, Isaak and Henry Zimmerman Macmillan Publishing Co., Inc. 866 Third Avenue, New York, N.Y. 10022. Through the complex chemistry of crosslinking, linkage of the amine residues of the recognizing substance and liposomes is established.

After the targeting agent is covalently attached to the lipid particle by way of covalent linkage to the cryoprotectant, or by way of covalent linkage to another targeting agent covalently linked to the cryoprotectant, the lipid particle may be lyophilized. The lipid particle may remain lyophilized prior to rehydration, or prior to rehydration and encapsulation of the agent of interest, for extended periods of time. In one embodiment, the lipid particle remains lyophilized for about 1 month, about 2 months, about 3 months, about 6 months, about 9 months, about 12 months, about 18 months, about 2 years or more prior to rehydration.

### Agent of Interest

The terms “encapsulation” and “entrapped,” as used herein, refer to the incorporation of an agent in a lipid particle. The agent is present in the aqueous interior of the lipid particle. In one embodiment, a portion of the encapsulated agent takes the form of a precipitated salt in the interior of the liposome. The agent may also self precipitate in the interior of the liposome.

For purposes of the present invention, “agent” means any agent or compound that can affect the body therapeutically, or which can be used in vivo for diagnosis. Examples of therapeutic agents include chemotherapeutics for cancer treatment, antibiotics for treating infections, antifungals for treating fungal infections, therapeutic nucleic acids including nucleic acid analogs, e.g., siRNA. In one embodiment, the agent of interest is a gene, polynucleotide, such as plasmid DNA, DNA fragment, oligonucleotide, oligodeoxynucleotide, antisense oligonucleotide, chimeric RNA/DNA oligonucleotide, RNA, siRNA, ribozyme, or viral particle. In one embodiment, the agent is a growth factor, cytokine, immunomodulating agent, or other protein, including proteins which when expressed present an antigen which stimulates or suppresses the immune system. In one embodiment, the agent is a diagnostic agent capable of detection in vivo following administration. Exemplary diagnostic agents include electron dense material, magnetic resonance imaging agents, radiopharmaceuticals and fluorescent molecules. Radionucleotides useful for imaging include radioisotopes of copper, gallium, indium, rhenium, and technetium, including isotopes ⁶⁴Cu, ⁶⁷Cu, ¹¹¹In, ^{99m}Tc, ⁶⁷Ga or ⁶⁸Ga. Imaging agents disclosed by Low et al. in U.S. Pat. No. 5,688,488, incorporated herein by reference, are useful in the liposomal complexes described herein.

In one preferred embodiment, the agent of interest is a nucleic acid, e.g., DNA, RNA, siRNA, plasmid DNA, short-hairpin RNA, small temporal RNA (stRNA), microRNA (miRNA), RNA mimetics, or heterochromatic siRNA. The nucleic acid agent of interest has a charged backbone that prevents efficient encapsulation in the lipid particle. Accordingly, the nucleic acid agent of interest may be condensed with a cationic polymer, e.g., PEI, polyamine spermidine, and spermine, or cationic peptide, e.g., protamine and polylysine, prior to encapsulation in the lipid particle. In one embodiment, the agent is not condensed with a cationic polymer.

In one embodiment, the agent of interest is encapsulated in the lipid particle in the following manner. The lipid particle, including a cryoprotectant and a targeting agent is provided lyophilized. The agent of interest is in an aqueous solution. The agent of interest in aqueous solution is utilized to rehydrate the lyophilized lipid particle. Thus, the agent of interest is encapsulated in the rehydrated lipid particle.

In one embodiment, two agents of interest may be delivered by the lipid particle. One agent is hydrophobic and the other is hydrophilic. The hydrophobic agent may be added to the lipid particle during formation of the lipid particle. The hydrophobic agent associates with the lipid portion of the lipid particle. The hydrophilic agent is added in the aqueous solution rehydrating the lyophilized lipid particle. An exemplary embodiment of two agent delivery is described below, wherein a condensed siRNA is encapsulated in a liposome and wherein a drug that is poorly soluble in aqueous solution is associated with the lipid portion of the lipid particle. As used herein, “poorly soluble in aqueous solution” refers to a composition that is less that 10% soluble in water.

Any suitable lipid: pharmaceutical agent ratio that is efficacious is contemplated by this invention. Preferred lipid: pharmaceutical agent molar ratios include about 2:1 to about 30:1, about 5:1 to about 100:1, about 10:1 to about 40:1, about 15:1 to about 25:1.

The preferred loading efficiency of pharmaceutical agent is a percent encapsulated pharmaceutical agent of about 50%, about 60%, about 70% or greater. In one embodiment, the loading efficiency for a hydrophilic agent is a range from 50-100%. The preferred loading efficiency of pharmaceutical agent associated with the lipid portion of the lipid particle, e.g., a pharmaceutical agent poorly soluble in aqueous solution, is a percent loaded pharmaceutical agent of about 50%, about 60%, about 70%, about 80%, about 90%, about 100%. In one embodiment, the loading efficiency for a hydrophobic agent in the lipid layer is a range from 80-100%.

In one aspect of the method, the liposome product is detectably labeled with a label selected from the group including a radioactive label, a fluorescent label, a non-fluorescent label, a dye, or a compound which enhances magnetic resonance imaging (MRI). In one embodiment, the liposome product is detected by acoustic reflectivity. The label may be attached to the exterior of the liposome or may be encapsulated in the interior of the liposome.

Pharmaceutical compositions using the lipid particles of the present invention can be administered by any convenient route, including parenteral, enteral, mucosal, topical, e.g., subcutaneous, intravenous, topical, intramuscular, intraperitoneal, transdermal, rectal, vaginal, intranasal or intraocular. In one embodiment, the lipid particles of the present invention are not topically administered. In one embodiment, the delivery is by oral administration of the particle formulation. In one embodiment, the delivery is by intranasal administration of the particle formulation, especially for use in therapy of the brain and related organs (e.g., meninges and spinal cord) that seeks to bypass the blood-brain barrier (BBB). Along these lines, intraocular administration is also possible. In another embodiment, the delivery means is by intravenous (i.v.) administration of the particle formulation, which is especially advantageous when a longer-lasting i.v. formulation is desired. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

It is still another object of the present invention to provide gene delivery using lipid particles as the gene delivery materials. For example, the mutant Raf gene can be targeted and delivered to tumor cells for anti-angiogenic purposes; the gene for the highly ctoxic cytokine TNF-alpha may be delivered to cancers to promote cell death; genes for the cytokines IL-12 and IFN-γ can be delivered to the lungs for allergy-induced hyperesponsiveness (AHR); and the cDNA for the glail cell ine derived neurogrowth factor (GDNF) may be targeted to the dopamine cells at the substantia nigra in Parkinson's disease patients.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

The invention is further illustrated by the following examples which should not be construed as limiting. The contents of all references cited throughout this application, including the U.S. provisional application 60/794,361 as well as the figures and table are incorporated herein by reference.

### EXAMPLE 1

### Layer-by-Layer Coating of Particles that Entrap and Deliver Drugs, Imaging Agents, Proteins and Nucleic Acids

### Methods

Liposome preparation and drug encapsulation. Liposome preparation and encapsulation was performed according to three-step process.

### Step 1

### Preparation of “Empty” (i.e., Drug-Free or Agent-Free) Regular Liposomes

Regular multilamellar liposomes (MLV) composed of PC:PE:CH at mole ratios of 3:1:1, were prepared by the traditional lipid-film method (Cattel et al. J Chemother. 2004, Suppl 4:94-7; Laverman et al. Crit Rev Ther Drug Carrier Syst. 2001; 18(6):551-66; Peer & Margalit. Arch. Biochem. Biophys 383 (2000) 185-190; Peer & Margalit. Neoplasia 6(4) (2004) 343-353). Briefly, the lipids were dissolved in chloroform-methanol (3:1, volume/volume), evaporated to dryness under reduced pressure in a rotary evaporator, and hydrated by the swelling solution that consisted of buffer alone (PBS), at the pH of 7.2. This was followed by extensive agitation using a vortex device and a 2-hour incubation in a shaker bath at 37° C. ULV were obtained by extrusion of the MLV, operating the extrusion device at 42-44° C. and under nitrogen pressures of 200 to 500 psi. The extrusion was carried out in stages using progressively smaller pore-size membranes, with several cycles per pore-size.

### Step 2

### Liposome Surface Modification (First Layer)

The modification was performed according to our previously reported process (Peer & Margalit. Arch. Biochem. Biophys 383 (2000) 185-190; Peer & Margalit. Neoplasia 6(4) (2004) 343-353; Peer et al. Biochim. Biophys. Acta. 1612 (2003) 76-82; Yerushalmi et al. Arch. Biochem. Biophys. 313 (1994) 267-273; Peer & Margalit et al. Inter. J. Cancer 108 (2004) 780-789). Briefly, hyaluronan (HA) was dissolved in water and preactivated by incubation with EDC, at pH 4 (controlled by titration with HCl) for 2 hours at 37° C. At the end of this step, the activated HA was added to a suspension of PE-containing liposomes in 0.1M borate buffer to a final pH of 8.6. The incubation with the liposomes was for 24 hours, at 37° C. At the end of the incubation, the liposomes were separated from excess reagents and by-products by centrifugation (135 k rpm, 1.5 hr, 4° C.) and repeated washings. The final ratio of ligand to lipid was 57 μg HA/μmole lipid.

### Surface Modification (Second Layer)

The antibody was dissolved first in PBS, pH 7.2. A 0.5-5 mg antibody/mL liposome suspension were added. Then 10 mg EDC/mL of lipid/antibody mixture was added. We reacted the mixture overnight at 4° C., then purified the conjugate by gel filtration using a column of sephadex G-75 or sepharose CL-4B column. The amount of antibody on the surface varied between preparations, different antibodies and different formulations.

### Step 3

### Drug or Agent Encapsulation in the Course of Liposome Reconstitution

Hyaluronan nanoliposomes (ULV-HA) or antibody-coated HA nanoliposomes (tHA) were reconstituted from the appropriate dried powder by rehydration with an aqueous (pure water) solution with or without a desire drug or agent. We took care to rehydrate back to the original pre-lyophilization liposome concentration, in order to retain the original buffering and salinity status. For the traditional method of preparation, we included the desire drug or agent in the swelling solution and used the same concentration in the washing buffers as well.

### Lyophilization

Lyophilization of liposome suspensions was performed on 1.0 ml aliquots. Samples were frozen for 2-4 hours at −80° C. and lyophilized for 48 hours. Reconstitution was to original volume using miliQ double distilled water with or without drug or agent.

### Drug Diffusion

The kinetics of drug diffusion were studied as previously described (Peer & Margalit. Arch. Biochem. Biophys 383 (2000) 185-190; Peer & Margalit. Neoplasia 6(4) (2004) 343-353; Peer et al. Biochim. Biophys. Acta. 1612 (2003) 76-82; Yerushalmi et al. Arch. Biochem. Biophys. 313 (1994) 267-273; Peer & Margalit et al. Inter. J. Cancer 108 (2004) 780-789). Briefly, a suspension of liposomes (0.5-1.0 ml) was placed in a dialysis sac and the sac was immersed in a continuously-stirred receiver vessel, containing drug-free buffer (phosphate buffered saline at pH 7.2). Receiver to liposome sample volume ratios were in the range of 10-16. At designated periods, the dialysis sac was transferred from one receiver vessel to another containing fresh (i.e., drug-free) buffer. Drug concentration was assayed in each dialysate and in the sac (at the beginning and end of each experiment).

In order to obtain a quantitative evaluation of drug release, experimental data were analyzed according to a previously-derived multi-pool kinetic model (Peer & Margalit. Arch. Biochem. Biophys 383 (2000) 185-190; Peer & Margalit. Neoplasia 6(4) (2004) 343-353; Peer et al. Biochim. Biophys. Acta. 1612 (2003) 76-82; Yerushalmi et al. Arch. Biochem. Biophys. 313 (1994) 267-273; Peer & Margalit et al. Inter. J. Cancer 108 (2004) 780-789). For this model, the relationship between time (the free variable) and the dependant variable f(t)—the cumulative drug released into the dialysate at time t, normalized to the total drug in the system at time=0—is expressed in equation 1 (below), where fj is the fraction of the total drug in the system occupying the jth pool at time=0, and kj is the rate constant for drug diffusion from the j'th pool.

$$f ( t ) = {\sum }_{j = 1}^{n} {f}_{j} ( 1 - {exp}^{- {k}_{j} t} ) \qquad Equation 1$$

### Encapsulation Efficiency

Defined as the ratio of entrapped drug or agent to the total drug or agent in the system, encapsulation efficiency can be determined by two independent methods:

(1) By centrifugation. Samples of complete liposome preparation (i.e., containing both encapsulated and unencapsulated drug) are centrifuged as described above. The supernatant, containing the unencapsulated drug, is removed and the pellet, containing the liposomes with encapsulated drug, is resuspended in drug free buffer. The drug is assayed in the supernatant and in the pellet, as well as in the complete preparation, from which the encapsulation efficiency and conservation of matter can be calculated.

(2) From data analysis of efflux kinetics. As discussed above, data analysis yields the parameter fj. When the efflux experiment is performed on samples from the complete liposome preparation, the magnitude of fj for the pool of encapsulated drug is also the efficiency of encapsulation.

### 3-(4,5-Dimethylthiazol-2-yl)-2,5-diphenyl-2H-tetrazolium Bromide (MTT) Cytotoxicity Assay

K562 cells (either K562 mock cells or K562 Mac-1 cells, stably transfected with Mac-1 integrin) were grown in 1×RPMI supplemented with 10% FBS. Exponentially growing cells were seeded at 2.5×10⁵/ml in 24-well plates and either left untreated or treated with graded dosages of free mitomycin (MMC) or equivalent doses of MMC formulated in liposomes. The treatment media was selected from the following MMC formulations: (i) the test system, MMC-loaded tHA-CBRM1/29, (ii) MMC-loaded ULV-HA, (iii) free MMC, (iv) free MMC dissolved in a suspension of empty tHA-CBRM1/29 and (v) free MMC dissolved in a suspension of empty ULV-HA. In addition, there were 3 drug-free controls, consisting of: (vi) no addition (i.e., only the serum-supplemented growth medium), (vii) empty tHA-CBRM1/29 and (viii) empty ULV. In all formulations, the solvent was serum-supplemented cell growth medium. Cells were washed twice with Hepes-buffered saline (HBS) then re-incubated with drug-free media for additional 48 hours. Cells were spun down and re-suspended in 0.5 ml of MTT containing medium (0.5 mg/ml), and incubated at 37° C. for 1 h. Cells were spun down and re-suspended in 1 ml of 0.04 N HCl in isopropyl alcohol to lyse the cells. After 5 min at room temperature, samples were spun down and 250-μl aliquots were used for absorbance measurements. Cell viability was measured as the difference in the absorbance 550 and 620 nm. The assay detects living, but not dead cells. Data are expressed as a percent of the control healthy growing cells.

### Results

Hyaluronan Acts as a Cryoprotectant for Nanoliposomes Coated with Monoclonal Antibodies

Coating with a cryoprotectant provide structural protection to the nanoliposomes. Table I shows a layer-by-layer coating with hyaluronan as a cryoprotectant before lyophilization and reconstitution in drug free water.

TABLE I
Layer-by-Layer Coating of Nanoliposomes
Before Lyophilization

| PARTICLE NAME | Size (nm) | Zeta Potential (mV), pH 7.4 |
|---|---|---|
| RL | 85 ± 12 | 0.25 ± 0.01 |
| ULV | 82 ± 15 | 0.3 ± 0.02 |
| ULV-HA | 105 ± 12 | −15.9 ± 0.5 |
| tHA-TS 2/4 | 135 ± 25 | 0.8 ± 0.2 |
| tHA-CBRM1/5 | 138 ± 35 | 0.7 ± 0.2 |
| tHA-CBRM1/29 | 128 ± 28 | 1.1 ± 0.3 |
| tHA-IgG 57 | 122 ± 30 | 1.3 ± 0.6 |
| tHA-TS1/22 | 140 ± 35 | 1.4 ± 0.2 |
| tHA-Her2 | 115 ± 20 | 4.8 ± 2.2 |
| tHA-IgG1 control isotype | 131 ± 29 | 1.8 ± 0.5 |

Measurements were done with Zetasizer nano SZ, instrument (Malvern, UK). Abbreviations: RL—regular (non-targeted) liposomes composed from phosphatidylcholine (PC) and cholesterol (CH) at a molar ratio of 7:3. ULV—unilamelar vesicles (ULV) composed of PC:CH and dipalmitoyl-phosphatidylethanolamine (DPPE) at a molar ratio of 3:1:1. ULV-HA—ULV made same as ULV (PC:DPPE:CH (3:1:1)), covalently linked to hyaluronan (HA), coating density (final): 57 μg HA/μmol lipid. tHA-TS 2/4—targeted HA ULV (same as ULV-HA, in composition and coating density) and covalently linked (amine to carboxyl) to antibody against human integrin LFA-1 (TS 2/4), estimated 89 molecules of antibody/particle. tHA-CBRM1/5—same as above (tHA—in composition and coating density) and covalently linked to antibody against human integrin Mac-1 (CBRM1/5), estimated 120 molecules of antibody/particle. tHA-CBRM1/29—same as above (tHA—in composition and coating density) and covalently linked to antibody against human integrin Mac-1 (CBRM1/29), estimated 105 molecules of antibody/particle. tHA-IgG57—same as above (tHA—in composition and coating density) and covalently linked to antibody against human integrin LFA-1 (IgG 57), estimated 110 molecules of antibody/particle. tHA-TS1/22—same as above (tHA- in composition and coating density) and covalently linked to antibody against human integrin LFA-1 (TS1/22), estimated 75 molecules of antibody/particle. tHA-Her2—same as above (tHA—in composition and coating density) and covalently linked to antibody against human ErbB2), estimated 130 molecules of antibody/particle. tHA-IgG1 control isotype—same as above (tHA—in composition and coating density) and covalently linked to antibody which is a human isotype control), estimated 102 molecules of antibody/particle. Note: The estimated antibody molecules per liposome ranged from 70 to 130, and were comparable among different immunoliposome types in a given experiment.

Table II shows the same systems after lyophilization: Formulations that lack the hyaluronan coating increased dramatically.

TABLE II
Layer-by-Layer Coating of Nanoliposomes After Lyophilization
and Reconstitution in Drug Free Water

| PARTICLE NAME | Size (nm) | Zeta Potential (mV), pH 7.4 |
|---|---|---|
| RL | 1320 ± 390 | 0.5 ± 0.2 |
| ULV | 1560 ± 450 | 0.45 ± 0.1 |
| ULV-HA | 134 ± 30 | −18.2 ± 1.4 |
| tHA-TS 2/4 | 125 ± 45 | 1.1 ± 0.5 |
| tHA-CBRM1/5 | 160 ± 55 | 1.2 ± 0.4 |
| tHA-CBRM1/29 | 145 ± 40 | 1.7 ± 0.5 |
| tHA-IgG 57 | 135 ± 37 | 1.2 ± 0.2 |
| tHA-TS1/22 | 167 ± 45 | 1.2 ± 0.4 |
| tHA-Her2 | 145 ± 28 | 4.3 ± 1.2 |
| tHA-IgG1 control isotype | 150 ± 35 | 1.8 ± 0.4 |

Looking at Table I and II it becomes clear that HA does provide a protection against lyophilization and reconstitution, thus only nano-scale liposomes that were covalently coated with HA have been structurally preserved.

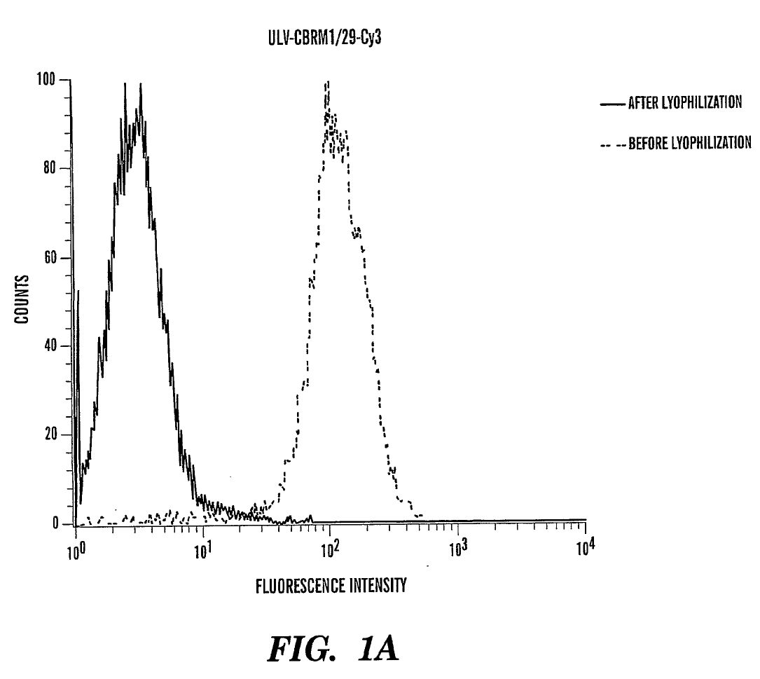

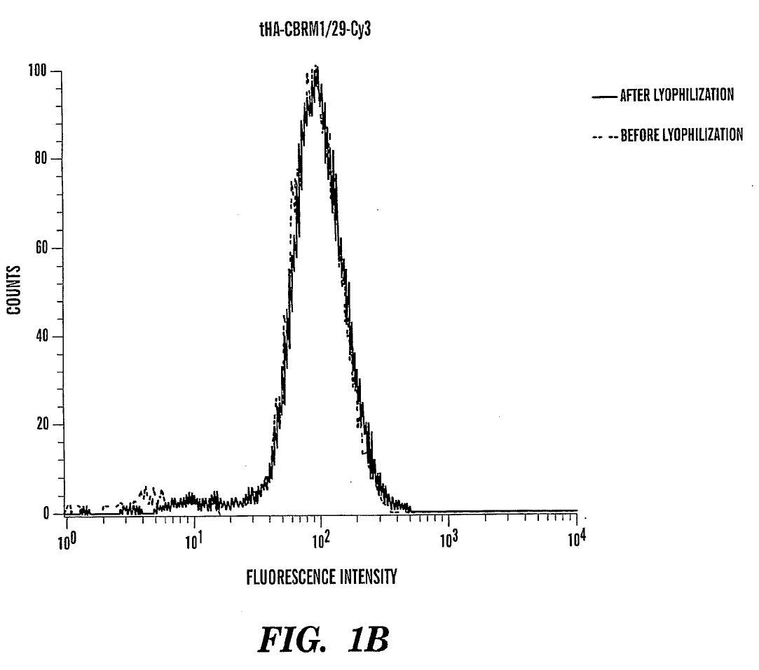

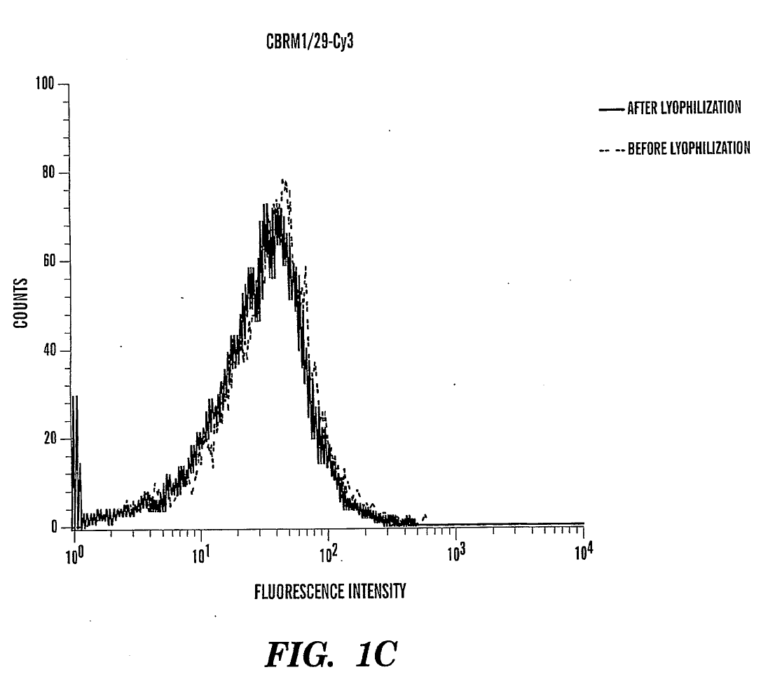

The structural preservation of nano-scale particles using a hyaluronan coat and an antibody on the surface can be further tested in a biological setting by immunofluorescence staining using flow cytometery. An example is shown in FIG. 1.

Antibody that binds to integrin Mac-1 or α_{M}β₂ (CBRM1/29; Diamond et al. Journal of Cell Biology, 120 (1993) 545-556.) was labeled with cy3 dye (Amersham Biosciences, UK) and incorporated into the surface of liposomes with HA coating (via a carbodiimide reagent, EDAC) and named tHA-CBRM1/29-cy3 or directly immobilized on the ULV surface (via Gluteraldehyde, GAD) and named ULV-CBRM1/29-cy3. Both nano-scale particles were tested in vitro using K562 human cell line stable transfected with integrin Mac-1.

FIG. 1A shows a dashed curve which represents binding of the nanoliposomes coated with CBRM1/29 (ULV-CBRM1/29-cy3) to K562 cells expressing Mac-1 integrin before lyophilization. The solid curve represents the binding of the carrier after lyophilization. As shown in this panel, mean fluorescence intensity before lyophilization was 175, and after lyophilization and reconstitution, 5.6. The fact that the carriers abolished binding upon lyophilization and reconstitution is an indication that structural damage to the liposome occurred during the lyophilization process due to the absence of cryoprotectant on the liposomal surface.

FIG. 1B shows an almost complete overlay of the dashed (MFI=161, before lyophilization) and solid (MFI=172, after lyophilization and reconstitute) curves of tHA-CBRM1/29-cy3 carriers. These carriers have HA as a cryoprotectant on their first layer, preserving their structure during lyophilization and reconstitution.

FIG. 1C shows a complete overlay between the curves on the antibody itself, CBRM1/29-cy3 (before (MFI=82) and after (MFI=73) lyophilization and reconstitution), showing that the changes in particles size are the major factor for changes in binding capacity to these cells.

### Functionality of Soluble Drugs Entrapped During Lyophilization and Reconstituted Inside Antibody Coated HA-Nanoliposomes

As an example of functionality maintained by the entrapment of soluble drugs inside antibody-coated HA-nanoliposomes, we entrapped mitomycin C (MMC). MMC is frequently first choice treatment for superficial-bladder and lung cancers, and a key component in combination chemotherapy for the treatment of breast, colorectal and prostate cancers (Carter et al. “Mitomycin C: current status and new developments.” New York: Academic Press, 1979; Hinoshita et al. Clin Cancer Res 2000; 6: 2401-7; Dalton et al. Cancer Res 1991; 51: 5144-52). In another therapeutic indication, it is applied as an antiproliferative agent to prevent abnormal healing of surgical wounds by slowing down excessive fibroblast proliferation, as in the case of glaucoma filtration surgery (Chang et al. J Ocul Pharmacol Ther 1998; 14: 75-95; Tahery et al. J Ocul Pharmacol 1989; 5: 155-179.).

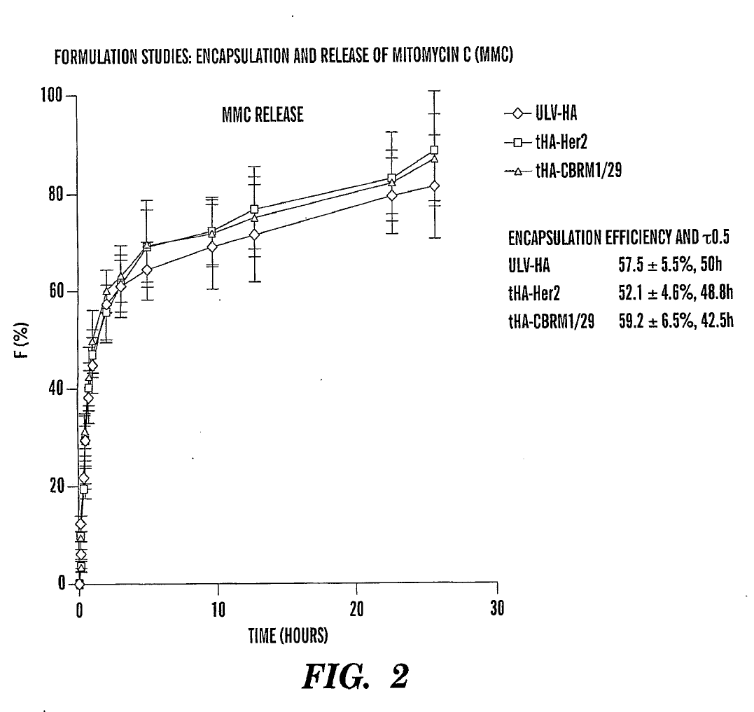

FIG. 2 presents the encapsulation and sustained release properties of HA-nanoliposomes having on their surface either CBRM1/29 antibody against integrin Mac-1 (tHA-CBRM1/29) or Her2 scFv against ErbB2 receptor (tHA-Her2).

The encapsulation efficiency of MMC in these carriers is between 52-59% with a half-life of drug efflux of approximately 50 hr. The advantage of quantifying the encapsulation efficiency is that one can separate the un-encapsulated drug from the encapsulated drug and give a homogeneous formulation. In other cases, where the entrapped drug is not highly toxic, like in the case of some antibiotics, two reservoirs (un-encapsulated, and encapsulated) could become an advantage—a first burst of non-encapsulated antibiotics and then a sustained release of it over a period of a week, for example.

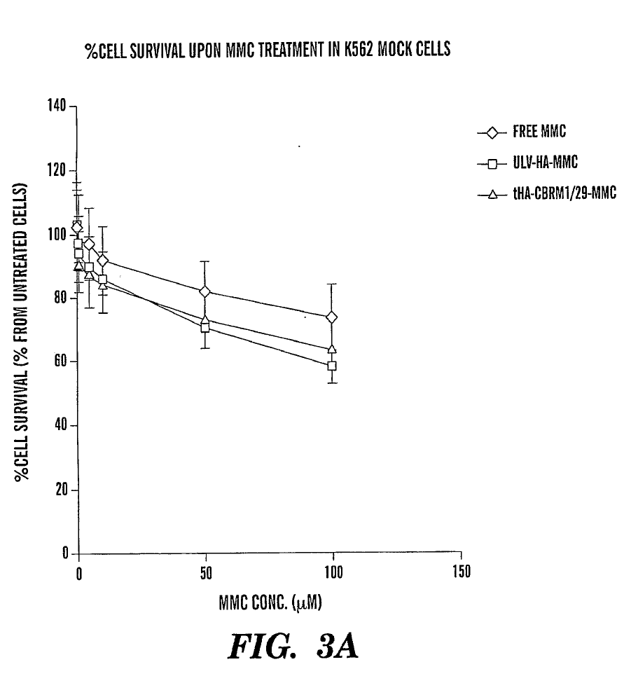

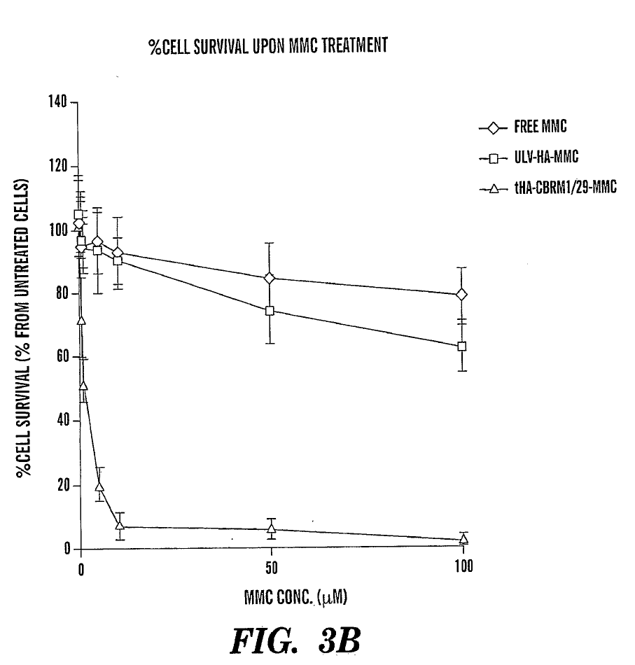

In the case of MMC, we removed the un-encapsulated MMC prior to using this formulation on cells expressing the integrin Mac-1 (K562 stable transfection with Mac-1 integrin), as indicated from FIG. 3. FIG. 3 represents a cytotoxicity assay made as described in the experimental section above. We plotted only three formulations. All the rest gave the same results as the free MMC formulation.

FIG. 3A represents all three formulations (free MMC, MMC entrapped in nano-scale HA-coated liposomes, named ULV-HA, and in antibody-coated HA-coated nanoliposomes (tHA-CBRM1/29)), in K562 mock cells. As clearly seen, there are no significant changes in the cell survival between these formulations. However, in FIG. 3B, when cells expressed integrin Mac-1, only the cells that got the liposomal formulation with the antibody had dramatic decrease in cell survival. Looking at the IC50 of the HA-coated nanoliposomes entrapping MMC compared to antibody-coated HA-nanoliposomes, we go from >100 μM in ULV-HA to 1.75 μM in tHA-CBRM1/29, about a 100-fold difference towards the antibody-mediated nano-scale targeting, showing an active targeting in vitro compare to the other formulations.

### EXAMPLE 2

### Micelles

Micelles are spherical colloidal nanoparticles into which many amphiphilic molecules self-assemble. In water, hydrophobic fragments of amphiphilic molecules form the core of a micelle, which may then be used as a cargo space for poorly soluble pharmaceuticals (Lasic, D. D. (1992) Nature 355, 279-280.; Muranishi, S. (1990) Crit. Rev. Ther. Drug Carrier Syst. 7, 1-33.). Hydrophilic parts of the molecules form the micelle corona. Micelle encapsulation increases bioavailability of poorly soluble drugs, protects them from destruction in biological surroundings, and beneficially modifies their pharmacokinetics and biodistribution (Hammad, M. A. & Muller, B. W. (1998) Eur. J. Pharmacol. Sci. 7, 49-55.). Because of their small size (usually 5-50 nm), micelles demonstrate spontaneous accumulation in pathological areas with leaky vasculature, such as infarct zones (Palmer, et al. 1984; Biochim. Biophys. Acta 797, 363-368) and tumors. This phenomenon is known as the enhanced permeability and retention effect.

Preparation of Nano-Micelles Coated with PEG or HA and Antibody

A lipid film was prepared by removing ethanol from the mixed solution of PEG2000-PE or DLPE under vacuum. To form micelles, the film was rehydrated at 65° C. in PBS, pH 7.4, and vortexed for 5 min. When DLPE was used we cross-linked it to HA as describe above using carbodiimide (EDAC). When required, 0.5 ml of a 0.5 mg of IgG1 or Her2 scFv were added to 0.5 ml of -PEG-PE-containing micelles or DLPE-HA micelles with carbodiimide for the DLPE-HA or GAD overnight at 4° C. As a control, we used DLPE without PEG or HA and cross-linked it to Her2 scFv or IgG1 via amine coupling (GAD). Then, the micelles were purified with a shepharose CL-4B column. The micelle size was measured by dynamic light scattering with a N4 Plus Submicron Particle System (Coulter) at a PEG-PE or DLPE-HA concentration of 2-10 mM.

Table III summarizes the results of the size distribution before and after lyophilization.

TABLE III
Size Distribution of Micelles Before and After Lyophilization

| | Before | After |
|---|---|---|
| Particle name | lyophilization (nm) | lyophilization (nm) |
| DLPE | 132 ± 20 | 1698 ± 435 |
| DLPE-HA | 189 ± 55 | 150 ± 34 |
| PEG-PE | 112 ± 30 | 167 ± 46 |
| DLPE-IgG1 | 159 ± 44 | 2320 ± 770 |
| DLPE-HA-IgG1 | 215 ± 55 | 200 ± 60 |
| PEG-PE-IgG1 | 155 ± 41 | 190 ± 60 |
| DLPE-Her2 (scFv) | 143 ± 32 | 2200 ± 560 |
| DLPE-HA-Her2 (scFv) | 195 ± 55 | 167 ± 70 |
| PEG-PE-Her2 (scFv) | 147 ± 30 | 170 ± 35 |

As one can see, PEG or HA can both preserve the structure of micelles when covalently coated with or without an antibody on the surface (be it a complete IgG or a scFv format). However, without the presence of HA or PEG there is no cryoprotection against lyophilization and reconstitute and size distribution are dramatically increased.

### EXAMPLE 3

### A Novel Platform for Entrapment of siRNAs and Other Genetic Materials in Particulate, Crystals, and Macroscopic Systems for Efficient Delivery and Targeting

### Methods

Labeling Antibodies with Fluorescence Dyes

Purified antibodies in PBS or HBS pH 7.4 without Tris, at a concentration between 0.5 to 2.0 mg/mL were used. Total volume was 1 mL for each labeling reaction. 1/10 volume of 1M NaHCO3, pH 8.5 was added to the antibody. The mixture (antibody/PBS/NaHCO3) was transfer to one vial of desiccated primary amine-reactive (succinimidyl esters) dye (Alexa 488, or cy3) and mixed well to dissolve the dye. The suspension was incubated at room temperature between 5-20 min (vary between antibodies) while protected from light. The reaction was quenched by adding ˜ 1/20 volume of 3M Tris, pH 7.2. The unlabeled dye was separated by a desalting column washed with PBS.

### Liposome Preparation

Lipids were from Avanti Polar lipids, Inc., AL, USA. Liposome preparation and encapsulation was performed according to three-step process.

### Step 1

### Preparation of “Empty” (i.e., Drug-Free) Regular Liposomes

Regular multilamellar liposomes (MLV) composed of PC:PE:CH at mole ratios of 3:1:1, were prepared by the traditional lipid-film method (Cattel et al. J Chemother. 2004, Suppl 4:94-7; Laverman et al. Crit Rev Ther Drug Carrier Syst. 2001; 18(6):551-66; Peer & Margalit. Arch. Biochem. Biophys 383 (2000) 185-190; Peer & Margalit. Neoplasia 6(4) (2004) 343-353.). Briefly, the lipids were dissolved in chloroform-methanol (3:1, volume/volume), evaporated to dryness under reduced pressure in a rotary evaporator, and hydrated by the swelling solution that consisted of buffer alone (PBS), at the pH of 7.2. This was followed by extensive agitation using a vortex device and a 2-hour incubation in a shaker bath at 37° C. To obtain multilamellar liposomes (MLV), we would have gone from this step directly to the next step. Nano-scale unilamellar liposomes (ULV) were obtained by extrusion of the MLV, with the extrusion device operated at 42-44° C. and under nitrogen pressures of 200 to 500 psi. The extrusion was carried out in stages using progressively smaller pore-size membranes, with several cycles per pore-size.

### Step 2

### Liposome Surface Modification (First Layer)

The modification was performed according to our previously reported process (Peer & Margalit. Arch. Biochem. Biophys 383 (2000) 185-190; Peer & Margalit. Neoplasia 6(4) (2004) 343-353). Briefly, hyaluronan (HA) was dissolved in water and preactivated by incubation with EDC, at pH 4 (controlled by titration with HCl) for 2 hours at 37° C. At the end of this step, the activated HA was added to a suspension of PE-containing liposomes in 0.1M borate buffer to a final pH of 8.6. The incubation with the liposomes was for 24 hours, at 37° C. At the end of the incubation, the liposomes were separated from excess reagents and by-products by centrifugation (135 k rpm, 1.5 hr, 4° C.) and repeated washings. The final ratio of ligand to lipid was 57 μg HA/μmole lipid.

### Targeting Surface Layer (Second Layer)

The antibody (unlabeled or fluorescently labeled, IgG57, TS1/22, KIM127—for integrin LFA-1; control isotype both human and mouse IgG1, CBRM1/5, CBRM1/29 for integrin Mac-1 and Her2 scFv for ErbB2 receptor) was dissolved first in PBS, pH 7.2. 0.5-5 mg antibody/mL liposome suspension were added. Then 10 mg EDC/mL of lipid/antibody mixture was added. We reacted the mixture over-night at 4° C. then purified the conjugate by gel filtration using a column of sephadex G-75 or sepharose CL-4B column. The amount of antibody on the surface vary between preparations, different antibodies and different formulations.

### Step 3

siRNAs Condensation and Encapsulation in the Course of Liposome Reconstitution

siRNAs were first condensed by polyethelenimine (PEI) or protamine (complete 51aa) at room temperature for 30 min to one hour. Hyaluronan nanoliposomes (ULV-HA) or antibody-coated HA nanoliposomes (tHA), as well as HA-liposomes (MLV-HA) or antibody-coated MLV-HA (MLV-HA-mAb) were reconstituted from the appropriate dried powder by rehydration with an aqueous (pure water) solution of the condensed siRNAs (PEI-siRNA) or siRNA-protamine. PEI was a linear 22KDa positively charge polymer (Exgen 500, Fermentas Life Sciences) or the Mr 25,000 branched form (Aldrich Chemical, Milwaukee, Wis.). Protamine (51aa, positively charged, natural protein) was from Abnova GmbH (Heidelberg, Germany). We took care to rehydrate back to the original pre-lyophilization liposome concentration, in order to retain the original buffering and salinity status.

### Lyophilization

Lyophilization of liposome suspensions was performed on 1.0 ml aliquots. Samples were frozen for 2-4 hours at −80° C. and lyophilized for 48 hours. Reconstitution was to original volume using miliQ double distilled water with condensed siRNA as described above.

### Encapsulation Efficiency

Defined as the ratio of entrapped siRNA to the total siRNA in the system, encapsulation efficiency can be determined by centrifugation. Samples of complete liposome preparation (i.e., containing both encapsulated and unencapsulated siRNA) were centrifuged as described above. The supernatant, containing the unencapsulated siRNA, is removed and the pellet, containing the liposomes with encapsulated siRNA, is resuspended in siRNA free buffer. siRNA is assayed in the supernatant and in the pellet, as well as in the complete preparation, from which the encapsulation efficiency and conservation of matter can be calculated.

Preparation of siRNAs

siRNAs with the following sense and antisense sequences were used:

CD4,

(sense; SEQ ID NO: 1)

5′-P.GAUCAAGAGACUCCUCAGUUU-3′;

(antisense; SEQ ID NO: 2)

5′-P.ACUGAGGAGUCUCUUGAUCUU-3′;
Luciferase-cy3 labeled,

(sense SEQ ID NO: 3)

5′-cy3-CGUACGCGGAAUACUUCGAdTdT-3′;

(antisense; SEQ ID NO: 4)

5′-UCGAAGUAUUCCGCGUACGdTdT-3′;
Luciferase,

(sense; SEQ ID NO: 5)

5′- CGUACGCGGAAUACUUCGAdTdT-3′;

(antisense; SEQ ID NO: 6)

5′-UCGAAGUAUUCCGCGUACGdTdT-3′.

All siRNAs were synthesized by Dharmacon Inc., using 2′-ACE protection chemistry. The siRNAs strands were deprotected and annealed according to the manufactur's instructions.
siRNA Detection

siRNA-cy3 or FITC labeled from Dharmacon, Inc. have been used for detection of encapsulated matter. For non fluorescence siRNA (CD4—targeted siRNA) or (luciferase targeted siRNA), both from Dharmacon, Inc. we used the Qant-iT™ RiboGreen® Assay from Molecular Probes.

Co-Encapsulation of Poorly Soluble Drug and siRNA in Different Phases

For entrapping a poorly-soluble drug we used Taxol (TX)—Paclitaxel from Taxus Yannanesis, sigma at a concentration of 2 mg/mL (˜2.34 mM). The lipid composition included PC:CH:DPPE at a mole ratio of 3:1:1 and a lipid concentration of 78 mM. Lipids were dissolved in ethanol 100% as well as taxol, and were stirred at 42° C. for 20 min. we added 5 μCi of 3H-TX (American radio chemicals) to the mixture. The TX was added in a concentration that represented 3% mole with respect to phospholipids, which is an optimal concentration for the stability of TX inside the liposomes (Stevens et al. Pharmaceutical Research, 21 (12) (2004) 2153-2157). The mixture then was evaporated under vacuum and incubated with borate buffer (0.1M, pH 9.0) for 2 hr at 65° C. in order to form liposomes. Then the liposomes were extruded at the same temperature under nitrogen pressures of 200 to 500 psi. The extrusion was carried out in stages using progressively smaller pore-size membranes, with several cycles per pore-size. The rest of the process (included HA coating and antibody coated) were done as describe above for nanoliposomes. siRNA entrapment was also done as describe above. TX encapsulation efficiency and sustained release was measured using the radiolabel trace and analyzed according to our previously reported drug diffusion experiments.

### Drug Diffusion

The kinetics of drug diffusion was studied as previously described (Stevens et al. Pharmaceutical Research, 21 (12) (2004) 2153-2157; Carman et al. J. Immunology, 171 (2003) 6135-6144; Novina et al. Nature Med. 2002 8(7) 681-6; Fraemohs et al. J Immunol. 2004 173(10):6259-64; Huang et al. (2006) J. Leukoc. Biol., 80(4): 905-14). Briefly, a suspension of liposomes (0.5-1.0 ml) was placed in a dialysis sac and the sac was immersed in a continuously stirred receiver vessel, containing drug-free buffer (phosphate buffered saline at pH 7.2). Receiver to liposome sample volume ratios were in the range of 10-16. At designated periods, the dialysis sac was transferred from one receiver vessel to another containing fresh (i.e., drug-free) buffer. Drug concentration was assayed in each dialysate and in the sac (at the beginning and end of each experiment).

In order to obtain a quantitative evaluation of drug release, experimental data were analyzed according to a previously derived multi-pool kinetic model (Stevens et al. Pharmaceutical Research, 21 (12) (2004) 2153-2157; Carman et al. J. Immunology, 171 (2003) 6135-6144; Novina et al. Nature Med. 2002 8(7) 681-6; Fraemohs et al. J Immunol. 2004 173(10):6259-64; Huang et al. (2006, supra). For this model, the relationship between time (the free variable) and the dependant variable f(t)—the cumulative drug released into the dialysate at time t, normalized to the total drug in the system at time=0—is expressed in equation (1), where fj is the fraction of the total drug in the system occupying the jth pool at time=0, and kj is the rate constant for drug diffusion from the j′th pool.

$$f ( t ) = {\sum }_{j = 1}^{n} {f}_{j} ( 1 - {exp}^{- {k}_{j} t} ) \qquad Equation 1$$

### Separation of Human Blood

Human neutrophils or lymphocytes were isolated from freshly drawn blood as described (Carman et al. J. Immunology, 171 (2003) 6135-6144). CD4 blasts were generated by isolating CD4+ T cells from peripheral blood lymphocytes of normal donors by immunomagnetic beads (Miltenyi Biotech) as previously reported (Novina et al. Nature Med. 2002; 8(7): 681-6) and culturing them in RPMI 1640 containing 10% FCS for 3 days in the presence of 4 μg/mL phytohemagglutinin (PHA), then expend the cells with the same media (without PHA) but with IL-2 at 10 ng/mL for 3 more days.

siRNA Transfection

siRNA transfections were preformed with different formulations of siRNA targeted against human CD4, siRNA-luciferase, or siRNA-cy3. As a positive control we used Fugene 6 and ExGen 500 (PEI) transfection reagents. Cells were seeded in 24 or 96 well plates at a density of 5000−2×10⁵ cells/well. For each experiment, several controls have been used, including: siRNA alone, siRNA formulated with commercial transfected reagent (PEI or Fugene 6), HA-liposomes without an antibody on their surface entrapping siRNA condensed with PEI or protamine, siRNA entrapped in tHA-IgG1 (irrelevant antibody, mouse or human control isotype), siRNA entrapped in tHA-TS1/22 and tHA-IgG57 (both for integrin LFA-1; Fraemohs et al. J Immunol. 2004 173(10):6259-64; Huang, et al. (2006, supra); Shimaoka et al. (2006) Proc. Natl. Acad. Sci. USA 103(38): 13991-6.) tHA-CBRM1/5 for integrin Mac-1 (Diamond et al. J Cell Biol. 1993; 120(2):545-56.). All the siRNAs entrapped in liposomes were first condensed with either PEI or protamine.

In silencing experiments, cells were incubated for 60 hr with various formulations in different conditions:

(1) non active conditions for integrin's activation: CaCl₂ and MgCl₂ 1 mM each;

(2) Active conditions for integrin's activation: (a) MgCl₂ 5 mM, EGTA 1 mM, and CBRLFA1/2 (activating antibody) at 10 μg/mL; (b) MnCl₂ 1 mM; (c) PMA at 100 nM; (d) TNF-α at 2 ng/mL (when neutrophils have been used).

For physiological conditions: plates were pre-coated with ICAM-1 (10 μg/mL); SDF-1 (5 μg/mL); or both; anti-CD3 (10 μg/mL), or a combination of anti-CD3 and ICAM-1 (at the same concentration listed above). Coating buffer: 20 mM Tris pH 9.0, NaCl 150 mM.

60 hr later, cells were harvested and a flow cytometry analysis using anti-CD4-FITC labeled and separately, anti-LFA-1 antibody—Alexa 488 or Cy3-labeled was perform to determine the silencing properties of CD4 using the liposomal system.

### Flow Cytometry

FITC-conjugated α CD4, Alexa 488-conjugated α LFA-1 (TS2/4, TS1/18), and cy3-conjugated siRNA were used for staining. Data were acquired and analyzed on FACSan with CellQuest software (Becton Dickinson, Franklin Lakes, N.J.).

### Confocal Analysis

Confocal analysis was done with human primary neutrophils activated with TNF-α (2 ng/mL). cy3-siRNA (luciferase) was condensed by 10 μg/mL protamine and entrapped in tHA-CBRM1/5 system.

An antibody to LFA-1 (TS2/4-Alexa 488) was used to stain the membrane of these cells. Confocal imaging was performed with a Bio-Rad Radiance 2000 laser-scanning confocal system (Bio-Rad, Hercules, Calif.) on an Olympus BX50BWI microscope (Melville, N.Y.) with a ×100 water immersion objective.

### Results

### Preparation of the Carriers and Characterization

Four different carriers have been prepared by a layer-by-layer coating method that was described in the method section. Table IV show physical characteristics of these four different carriers.

TABLE IV
physical parameters for of carriers of siRNA

| | Size | Zeta Potential |
|---|---|---|
| Carrier name | (diameter in nm) | (mV) in pH 7.4 |
| ULV-HA | 105 ± 12 | −15.9 ± 0.5 |
| tHA-IgG1 (mouse) | 125 ± 34 | 1.3 ± 0.3 |
| tHA-IgG1 (human) | 131 ± 29 | 1.8 ± 0.5 |
| tHA-CBRM1/5 | 138 ± 35 | 0.7 ± 0.2 |
| tHA-IgG57 | 122 ± 30 | 1.3 ± 0.6 |
| tHA-TS1/22 | 140 ± 35 | 1.4 ± 0.2 |

Measurements were done with Zetasizer nano SZ, instrument (Malvern, UK). Abbreviations: ULV-HA—liposomes were made from phosphatidylcholine (PC), Cholesterol (CH) and dipalmitoyl-phosphatidylethanolamine (DPPE) at a molar ratio of 3:1:1. covalently linked to hyaluronan (HA). coating density (final):57 μg HA/μmol lipid. tHA-IgG1 control isotype—same as above (tHA—in composition and coating density) and covalently linked to antibody which is a human isotype control), estimated 102 molecules of antibody/particle. tHA-CBRM1/5—same as above (tHA—in composition and coating density) and covalently linked to antibody against human integrin Mac-1 (CBRM1/5), estimated 120 molecules of antibody/particle. tHA-IgG57—same as above (tHA—in composition and coating density) and covalently linked to antibody against human integrin LFA-1 (IgG 57), estimated 110 molecules of antibody/particle. tHA-TS1/22—same as above (tHA—in composition and coating density) and covalently linked to antibody against human integrin LFA-1 (TS1/22), estimated 75 molecules of antibody/particle. Note: The estimated antibody molecules per liposome ranged from 70 to 130, and were comparable among different immunoliposome types in a given experiment.

A basic requirement for a siRNA carrier is to have a highly selective marker on its surface that will cause internalization of the carrier and its cargo into the cells. So for each antibody (CBRM1/5, IgG57, and TS1/22)—we verified that these antibody bind to cells as well as deliver siRNA to specific cell type.

### Binding of Immunonanoliposomes to Their Target Cells

In order to determine if an antibody that recognize specifically the active conformation of integrin Mac-1 (α_{M}β₂), expressed solely on leukocytes, and immobilized on a nano-scale liposomes, could still target its receptor, CBRM1/5 was first labeled with cy3 dye as describe above in the method section and immobilized on the surface of the liposomes.

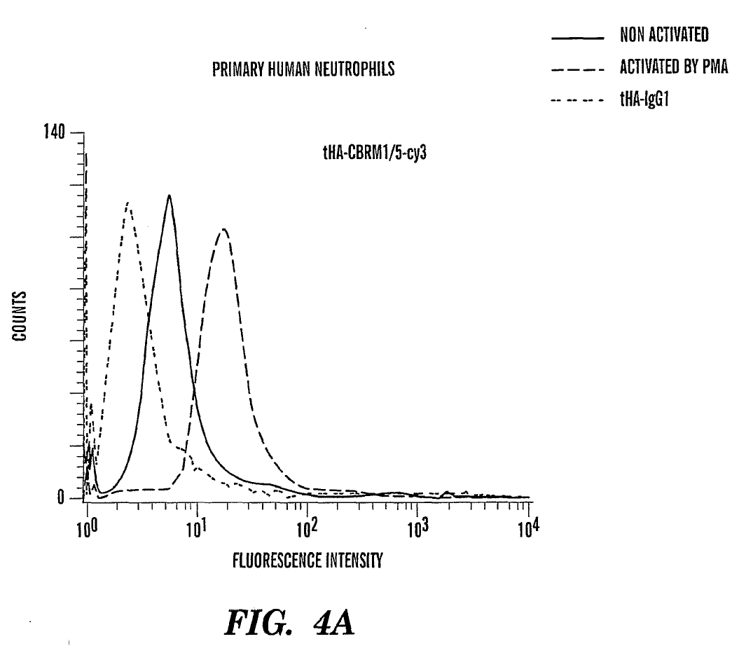

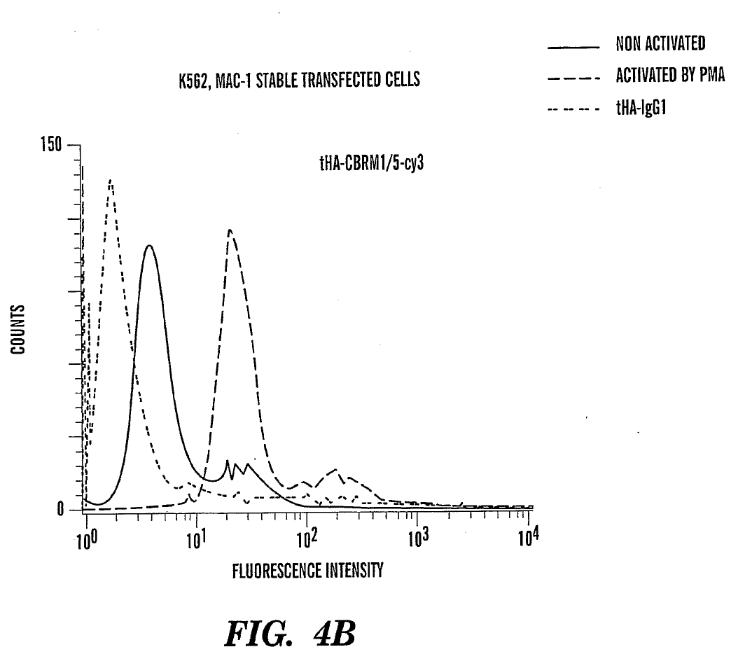

FIG. 4 shows binding of the nanoliposomes that immobilized CBRM1/5-cy3 on their surface in an active isolated primary human neutrophils (activation was done with TNF-α), or without activation. As indicated in FIG. 4, tHA-CBRM1/5-cy3 is bound to primary isolated human neutrophils only upon activation of the cells with TNF-α (FIG. 4A), whereas tHA-IgG1 mouse control isotype did not give any binding to these cells. Similar trends have been observed with other cells such as K562 cells stable transfected with Mac-1 integrin and activated by PMA (FIG. 4B).

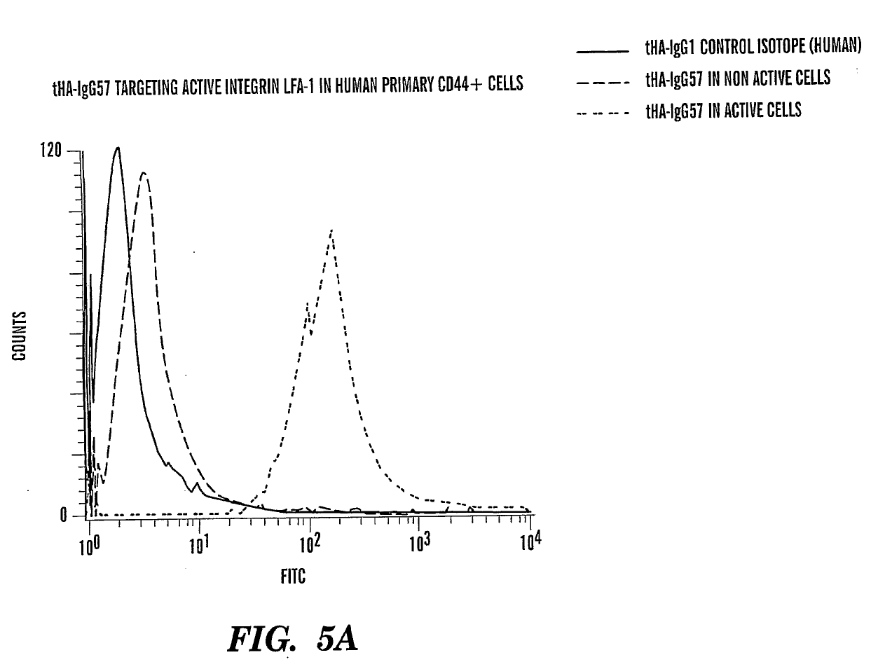

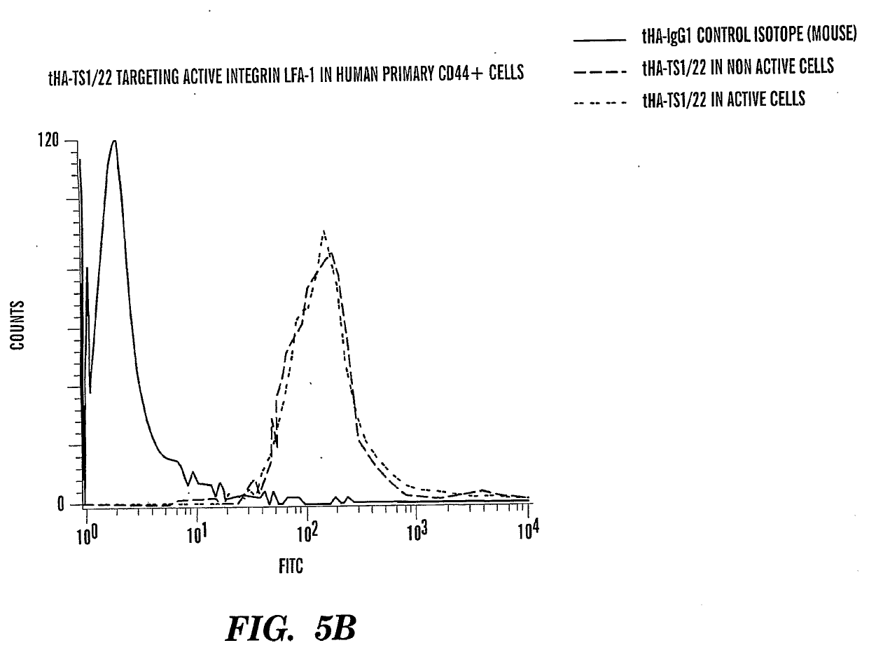

FIG. 5A, shows binding of IgG57 (tHA-IgG57) in an active/non active form of another integrin, LFA-1, in primary human lymphocytes compare to non binding curve by tHA-IgG1 (human isotype control). FIG. 5B shows binding with another antibody (TS1/22) on the surface of nanoliposomes. This antibody recognize both active and non active conformation of integrin LFA-1 that are overlay one on the other. As a negative control, a tHA-IgG1 mouse isotype control was used.

Formulations of siRNA Inside Carriers

siRNA was either formulated with protamine (51aa), PEI (linear or branched polymers) or alone for 1 hr at room temperature before incorporated into the liposomes in the course of reconstitution after lyophilization.

Table V represents the encapsulation efficiency that were calculated as listed in the experimental section.

TABLE V
formulation studies of siRNA entrapped
inside immunonanoliposomes

| | Efficiency of encapsulation (%) | | | |
|---|---|---|---|---|
| | | | PEI | |
| Carrier name | No condenser | Protamine | branched | PEI linear |
| ULV-HA | 25.5 ± 5.5 | 83.5 ± 10.1 | 92.0 ± 6.4 | 90.2 ± 8.9 |
| tHA-IgG1 | 32.0 ± 4.5 | 72.0 ± 6.7 | 85.6 ± 8.1 | 83.4 ± 7.7 |
| (mouse) | | | | |
| tHA-IgG1 | 27.4 ± 2.1 | 75.9 ± 4.4 | 97.8 ± 3.4 | 95.6 ± 4.4 |
| (human) | | | | |
| tHA-IgG57 | 28.3 ± 1.7 | 77.8 ± 4.4 | 95.9 ± 4.5 | 91.8 ± 3.4 |
| tHA-TS1/22 | 31.0 ± 3.1 | 76.9 ± 4.6 | 92.6 ± 6.1 | 94.9 ± 6.8 |
| tHA-CBRM1/5 | 29.8 ± 1.5 | 80.5 ± 4.3 | 97.3 ± 7.1 | 95.8 ± 5.5 |

The siRNA targeted CD4.

Luciferase and Luciferase-cy3 siRNA formulations gave the same trend.

Delivery of siRNA-Cy3 into Cells

In order to test the ability to deliver a fluorescently-labeled siRNA we have formulated siRNA-cy3 condensed with either PEI (linear) or protamine and entrapped it in tHA-CBRM1/5, tHA-IgG57, and tHA-TS1/22.

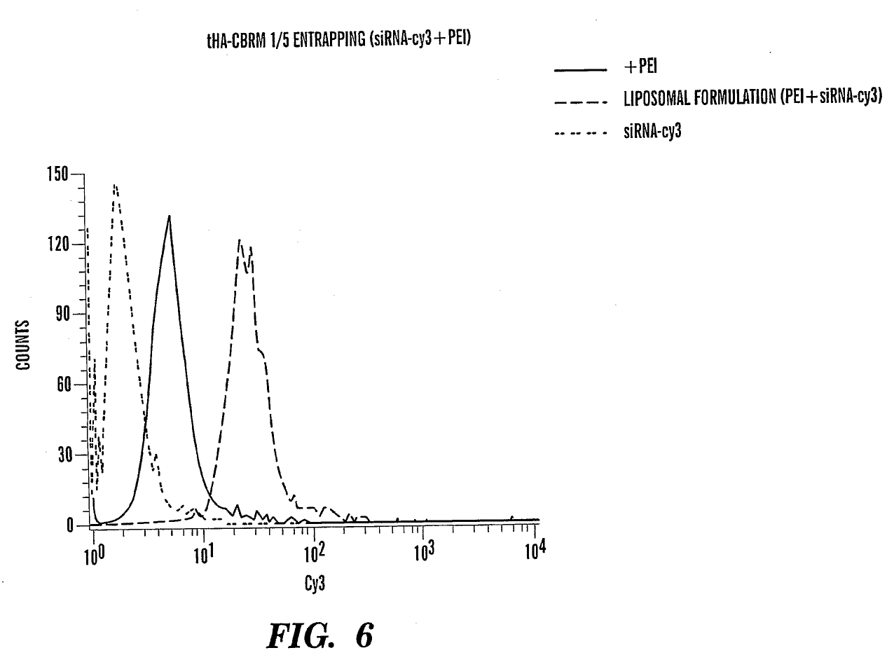

Cells expressing active Mac-1 integrin were positively targeted by tHA-CBRM1/5 entrapping siRNA-cy3 (condensed by linear PEI) (data not shown). FIG. 6 shows a flow cytometry analysis of Mac-1 WT cells with siRNA-cy3 alone, siRNA-cy3 transfected using linear PEI and siRNA-cy3 condensed by linear PEI and entrapped inside tHA-CBRM1/5 that target active integrin Mac-1.

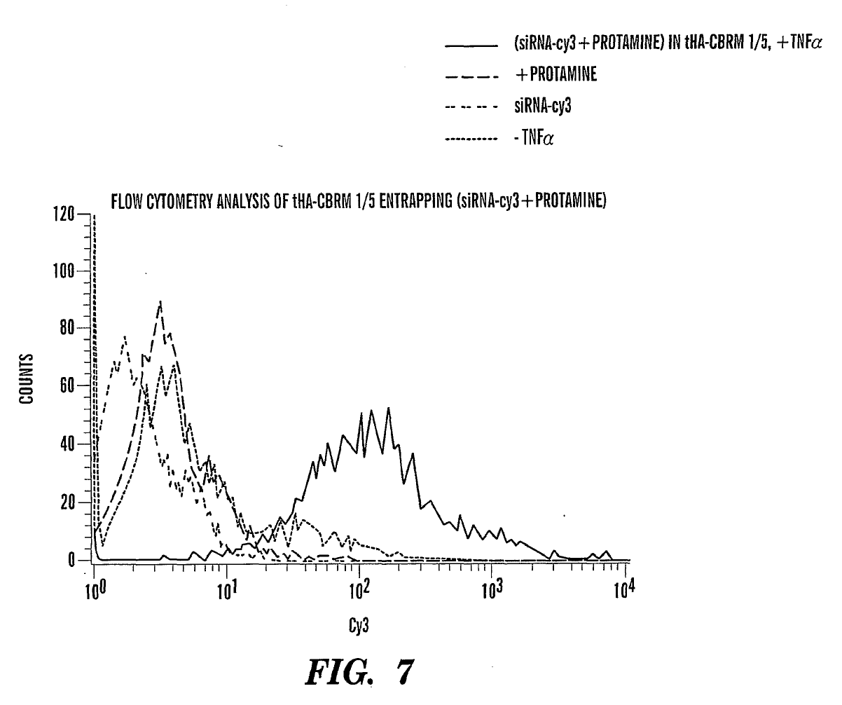

FIG. 7 shows flow cytometry analysis of siRNA-cy3 condensed by protamine and entrapped in tHA-CBRM1/5 in primary human neutrophils with or without activation of the cells.

The same trends were seen with siRNA (condensed either by protamine or linear PEI) entrapped inside tHA-TS1/22 and tHA-IgG57.

Co-Encapsulation of Poorly Soluble Drug and siRNA in Different Phases

We tested serum stability of immunonanoliposomes after co-entrapment of a poorly-soluble drug (in the organic phase) and a soluble drug (siRNA) in the hydrophilic phase and monitor the drug efflux from these particles in 50% human serum (Sigma). Analysis was performed as describe in the experimental section. Taxol (Paclitaxel) diffusion from the particles was monitored using radioactive trace of ³H-TX and siRNA-cy3 by its absorbance at 550 nm. The entrapment was preformed with three carriers bearing different antibodies on their surface: TS1/22, IgG57 (both against human integrin LFA-1); scFv(Her2) against human ErbB2 receptor, and as control systems we used ULV-HA (no antibody on the surface) and regular (conventional liposomes) (RL) composed of PC:CH 7:3.

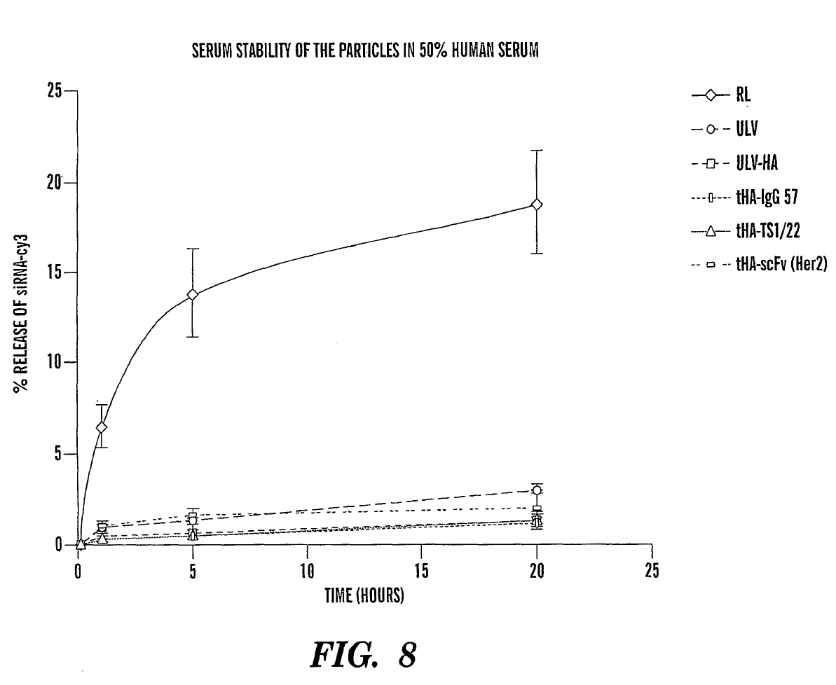

FIG. 8 shows the release profile of siRNA-cy3 from these carriers incubated for 20 hours in 50% human serum. Taxol release was negligible (<4%) in all the systems, indicating that these systems are highly stable at human serum.

This experiment shows the potential of a co-entrapment of drugs for different indications in the same carrier and their stability in serum.

Silencing Primary Human CD4+ Cells with Immunonanoliposomes Targeted to Integrin LFA-1

CD4-siRNA was formulated in tHA-IgG1, tHA-IgG57 (conformational sensitive delivery system) and tHA-TS1/22 (conformational insensitive delivery system) using protamine as its condenser as described in the experimental section above.

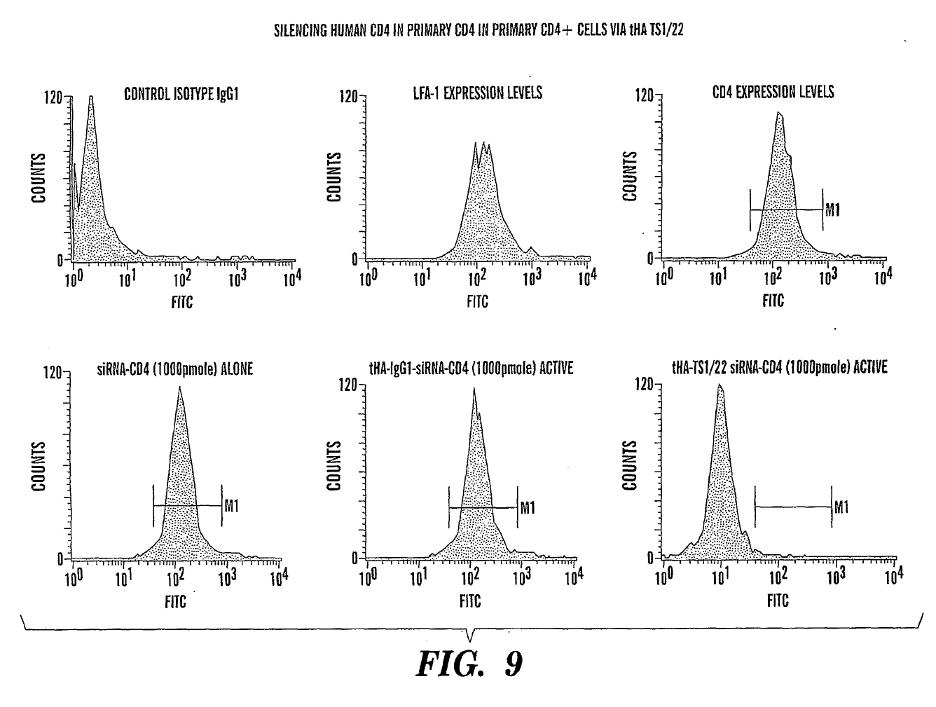

FIG. 9 shows selective silencing of CD4+ cells using tHA-TS1/22 that target integrin LFA-1 on these cells. Looking it FIG. 9, it becomes clear that tHA-TS1/22 is highly effective in specific delivery of siRNA into the cells.

Primary T cells are highly resistance to transfection (Zhao Y, Zheng Z, Cohen C J, Gattinoni L, Palmer D C, Restifo N P, Rosenberg S A, Morgan R A. High-Efficiency Transfection of Primary Human and Mouse T Lymphocytes Using RNA Electroporation. Mol Ther. 2005 Sep 1 [Epub ahead of print]; Lee et al. Blood. 2005, 106(3):818-26) and many non-traditional methods have been developed in order to show effective delivery of genetic material. However, the ability to effectively deliver siRNA and other genetic matter such as plasmid DNA is still unsolved.

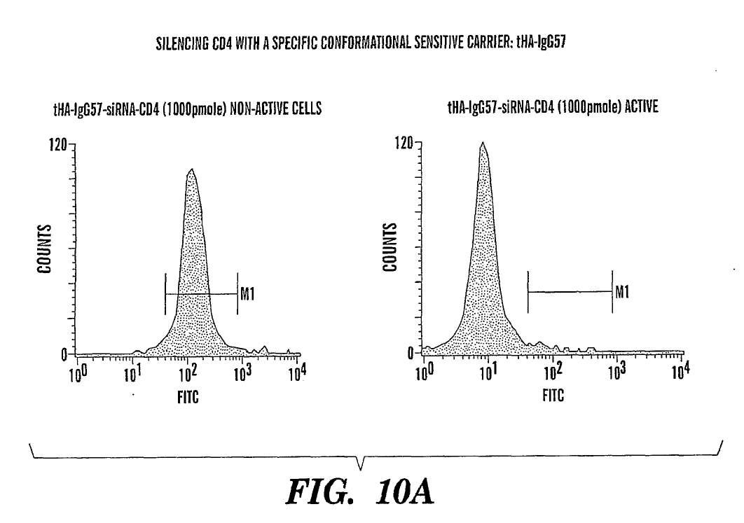

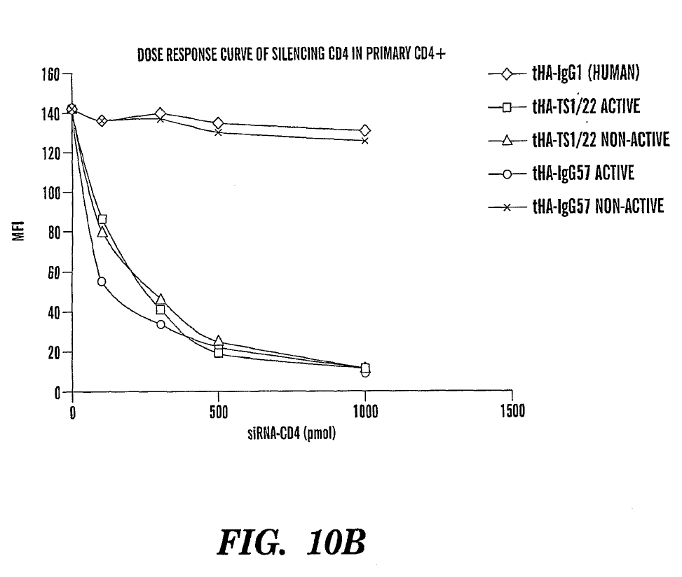

FIG. 10 shows effective silencing in the same cells with a different delivery system targeting only active integrin (tHA-IgG57). A dose response of siRNA-CD4 is shown in FIG. 10B. From this figure, one clearly see that only activated cells targeted by either tHA-TS1/22 or tHA-IgG57 can effectively deliver and silence CD4, whereas in non active cells, the conformation insensitive system, tHA-TS1/22 can deliver siRNA, but not the conformational sensitive delivery system (tHA-IgG57).

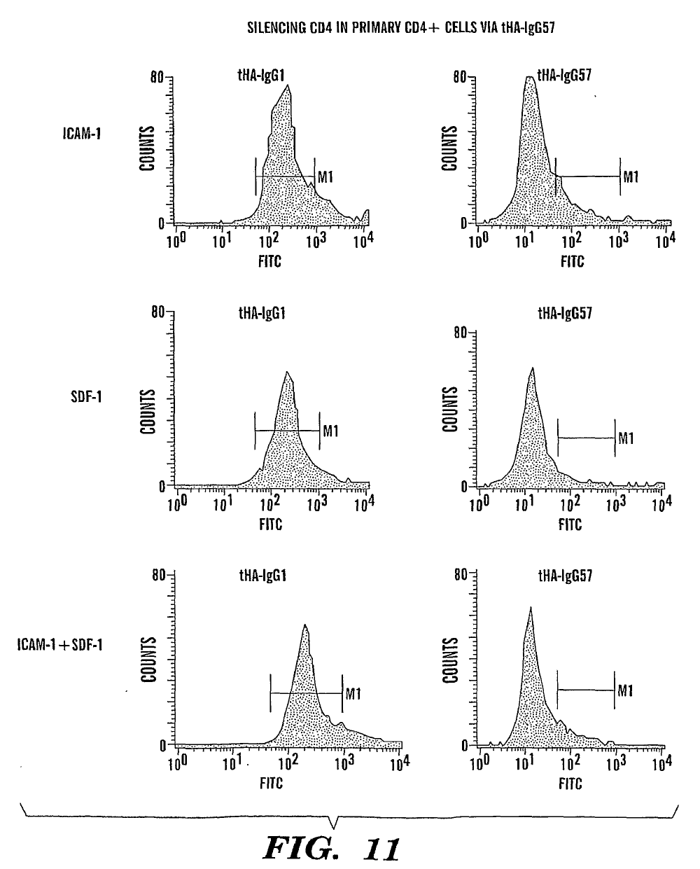

FIG. 11 describes CD4 silencing in physiological conditions (in an inflammation in vitro model). ICAM-1, SDF-1 and both were immobilized on 96 well plates as described in the experimental section. Maximal silencing have occurred using ICAM-1 (a ligand for integrin LFA-1 that only binds when cells are active, i.e., ready for this receptor-ligand interaction) and SDF-1 (a cytokine) have been immobilized together on 96 well plates.

This is an efficient simulation of inflamed areas, showing again the high selectivity of these delivery systems.

### EXAMPLE 4

### siRNAs Entrapped and Specifically Targeted to Active Integrins Using Immunomicelles

Micelles are spherical colloidal nanoparticles into which many amphiphilic molecules self-assemble. In water, hydrophobic fragments of amphiphilic molecules form the core of a micelle, which may then be used as a cargo space for poorly soluble pharmaceuticals (Lasic, D. D. (1992) Nature 355, 279-280; Muranishi, S. (1990) Crit. Rev. Ther. Drug Carrier Syst. 7, 1-33). Hydrophilic parts of the molecules form the micelle corona. Micelle encapsulation increases bioavailability of poorly soluble drugs, protects them from destruction in biological surroundings, and beneficially modifies their pharmacokinetics and biodistribution (Hammad, M. A. & Muller, B. W. (1998) Eur. J. Pharmacol. Sci. 7, 49-55.). Because of their small size (usually 5-50 nm), micelles demonstrate spontaneous accumulation in pathological areas with leaky vasculature, such as infarct zones (Palmer, et al. (1984) Biochim. Biophys. Acta 797, 363-368.) and tumors (Gabizon, A. A. (1995) Adv. Drug Delivery Rev. 16, 285-294; Yuan, et al. (1994) Cancer Res. 54, 3352-3356). This phenomenon is known as the enhanced permeability and retention effect

Preparation of Nano-Micelles Coated with PEG or HA and Antibody

Lipids were from AvantiPolar Lipids, Inc., AL, USA. A lipid film was prepared by removing ethanol from the mixed solution of PEG2000-PE or DLPE under vacuum. To form micelles, the film was rehydrated at 65° C. in PBS, pH 7.4, and vortexed for 5 min. When DLPE was used we cross-linked it to HA as describe in example 3 using carbodiimide (EDAC). When required, 0.5 ml of a 0.5 mg of IgG1 or TS1/22 (IgG1) against human integrin LFA-1, were added to 0.5 ml of -PEG-PE-containing micelles or DLPE-HA micelles with carbodiimide for the DLPE-HA or GAD overnight at 4° C. As a control, we used DLPE without PEG or HA and cross-linked it to Her2 scFv or IgG1 via amine coupling (GAD). The micelles were then purified with a shepharose CL-4B column. The micelle size was measured by dynamic light scattering with a N4 Plus Submicron Particle System (Coulter) at a PEG-PE or DLPE-HA concentration of 2-10 mM. Table VI summarizes the results of the size distribution of immuno micelles.

TABLE VI
size distribution of micelles

| Particle name | Before lyophilization (nm) |
|---|---|
| DLPE | 132 ± 20 |
| DLPE-HA | 189 ± 55 |
| PEG-PE | 112 ± 30 |
| DLPE-IgG1 | 159 ± 44 |
| DLPE-HA-IgG1 | 215 ± 55 |
| PEG-PE-IgG1 | 155 ± 41 |
| DLPE-TS1/22 | 143 ± 32 |
| DLPE-HA-TS1//2 | 195 ± 55 |
| PEG-PE-TS1/22 | 147 ± 30 |

Silencing CD4 in human primary CD4+ cells via immunomicelles. CD4-siRNA was formulated in PEG-PE-IgG1, DLPE-HA-IgG1 (as a negative control) and in DLPE-HA-TS1/22 and PEG-PE-TS1/22 (conformational insensitive delivery systems) that target integrin LFA-1 using protamine as its condenser as describe in the experimental section above for the liposomes.

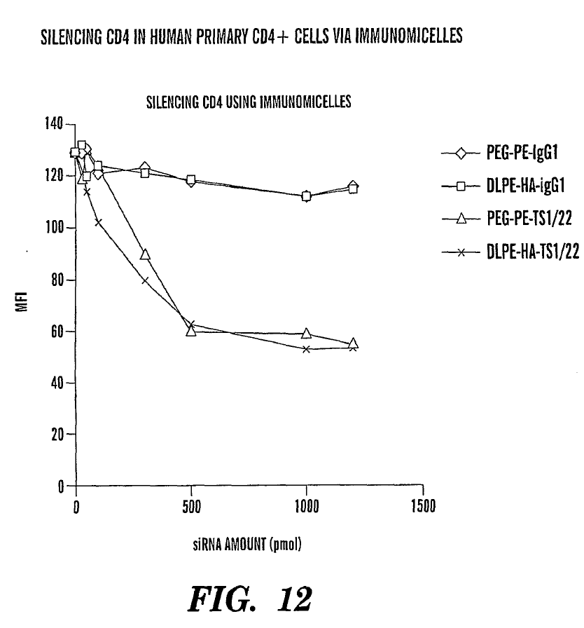

FIG. 12 shows a dose response curve starting at 30 pmol-1000 pmol CD4-siRNA. The data clearly shows a dose response curve that plateau at 500 pmol. We have demonstrated siRNA delivery using immunomicelles for the first time.

### EXAMPLE 5

### Stability of Lyophilized Immuno-HA-Nano-Liposomes Over 7 Months

The stability of the lyophilized immuno-nano-liposome powders was monitored for 7 months, using mitomycin C as the test drug. Table VII shows the size distribution and zeta potential at pH 7.4 of lyophilized powders that were resuspended with miliQ water at designated time points. As expected, and seen from Table VII, it is clear that regular non-modified liposomes cannot preserve their structure upon lyophilization and reconstitution. However, HA used as a cryoprotectant allows for structural preservation of the small ULV carriers over 7 months or longer. This is further supported by the results of mitomycin C entrapment, listed in Table VIII. As can be seen, both encapsulation efficiency and efflux kinetics are independent of the time-span during which the liposomal powder was stored prior to reconstitution with an aqueous solution of the drug.

TABLE VII
Regular and immuno-hyaluronan-nano-liposomes: the effect of
time from drying to rehydration on zeta potentials and on
liposome dimensions

| | Time span as | | | | |
|---|---|---|---|---|---|
| | lyophilized | Liposome | | | |
| | powder | diameter | | Zeta potential | |
| Liposome type | (days) | (nm) | | (mV)¹ | |
| Nano- | 0 | 101 | (±15)² | +2.7 | (±0.6) |
| Nano- | 2 | 1650 | (±670) | +4.1 | (±2.2) |
| CBRM1/29- | 0 | 120 | (±30) | −22.7 | (±4.5) |
| Hyaluronan-nano- | | | | | |
| CBRM1/29- | 2 | 155 | (±45) | −27.4 | (±5.5) |
| Hyaluronan-nano- | | | | | |
| CBRM1/29- | 90 | 142 | (±48) | −25.9 | (±6.1) |
| Hyaluronan-nano- | | | | | |
| CBRM1/29- | 210 | 161 | (±30) | −27.8 | (±3.3) |
| Hyaluronan-nano- | | | | | |

¹Measured at pH = 7.4;
²Each result is an average (and standard deviation) of 6 independent measurements.
CBRM1/29-targets integrin Mac-1.

TABLE VIII
Mitomycin C (MMC)-encapsulating-CBRM1/29-hyaluronan-
nano-liposomes: the effect of time from drying to rehydration
on encapsulation efficiency and drug efflux

| Time span as | Encapsulation | Efflux rate |
|---|---|---|
| lyophilized powder | efficiency | constant |
| (days) | (%) | (hours⁻¹ * 1000) |
| 0 | 44.6 (±2.0)¹ | 32.8 (±4.2) |
| 90 | 41.8 (±1.6) | 31.2 (±3.3) |
| 210 | 41.8 (±0.1) | 31.2 (±0.01) |

¹Each result is an average (and standard deviation) of 3 independent measurements.

The references cited herein and throughout the specification are incorporated herein by reference.

1. A method for layer by layer coating of lipid particles with a cryoprotectant and a targeting moiety, comprising the steps of:
(a) providing a lipid particle having phospholipids with a functional group wherein the functional group is available for crosslinking to a cryoprotectant;
(b) crosslinking the cryoprotectant to the functional group; and
(c) crosslinking a targeting moiety to the cryoprotectant on the lipid particle.

2. (canceled)

3. The method of claim 1, further comprising lyophilizing the lipid particle having the cryoprotectant and the targeting moiety covalently attached to it.

4. The method of claim 3, further comprising providing an aqueous solution of an agent and rehydrating the lyophilized lipid particle having the cryoprotectant and the targeting moiety crosslinked to it with the aqueous solution of the agent.

5. The method of claim 1, wherein the lipid particle is a liposome.

6. The method of claim 5, wherein the lipid particle is a micelle.

7. The method of claim 6, wherein the cryoprotectant is PEG.

8. The method of claim 1, wherein the cryoprotectant is a glycosaminoglycan.

9. The method of claim 8, wherein the cryoprotectant is hyaluronan.

10. The method of claim 4, wherein the agent is a nucleic acid.

11. The method of claim 10, wherein the nucleic acid is siRNA.

12. (canceled)

13. (canceled)

14. (canceled)

15. (canceled)

16. The method of claim 1, wherein the lipid particle comprises a hydrophobic agent in the lipid layer.

17. The method of claim 1, wherein the functional group is an amine group or a carboxyl group.

18. The lipid particle produced by the method of claim 1.

19. A composition comprising a lipid particle, a cryoprotectant and a targeting moiety, wherein the lipid particle comprises a phospholipid with a functional group, wherein the functional group is crosslinked to the cryoprotectant, the cryoprotectant is bound to the functional group of the phospholipids, and the targeting moiety is bound to the cryoprotectant.

20. The composition of claim 19, wherein the functional group is an amine group or a carboxyl group.

21. The composition of claim 19, wherein a hydrophilic agent is encapsulated in the lipid particle.

22. The composition of claim 19, wherein a hydrophobic agent is associated with lipids of the lipid particle.

23. The composition of claim 21, wherein the hydrophilic agent is selected from a group consisting of chemotherapeutic agents, antifungal compounds, genes or fragments of genes of proteins, RNA and fragments of RNA, antibody or functional fragments thereof, viral particles, radiopharmaceuticals, and magnetic resonance imaging agents.

24. The composition of claim 19, wherein the composition is lyophilized.

25. A method for encapsulating agents in a lipid particle, comprising the steps of:
(a) providing a lyophilized composition of claim 24;
(b) providing a hydrophilic agent in aqueous solution; and
(c) rehydrating the lyophilized composition with an aqueous solution comprising the hydrophilic agent.

26. The method of claim 25, wherein the composition is lyophilized in buffer for the hydrophilic agent and the aqueous solution is water.

27. The method of claim 25, wherein a hydrophobic agent is associated with the composition of step (a).

28. The method of claim 25, wherein the hydrophilic agent is a nucleic acid.

29. The method of claim 28, wherein the nucleic acid is siRNA.

30. (canceled)

31. (canceled)

32. (canceled)

33. (canceled)

34. A kit comprising a lyophilized cryoprotectant-conjugated lipid particle with a functional group that is available for crosslinking to a targeting agent.

35. The liposome kit of claim 34, wherein the cryoprotectant-conjugated lipid particles is further rehydrated and crosslinked to a targeting agent.

36. (canceled)

37. (canceled)

38. (canceled)

39. (canceled)

40. (canceled)

41. (canceled)

42. (canceled)

43. (canceled)

44. (canceled)

45. (canceled)
